(19) United States
(12) Patent Application Publication
(10) Pub. No.: US 2022/0287658 A1
(43) Pub. Date: Sep. 15, 2022

(54) IN VIVO IMMUNOIMAGING OF INTERLEUKIN-12

(71) Applicant: Wayne State University, Detroit, MI (US)

(72) Inventors: Nerissa Viola, Canton, MI (US); Heather Gibson, Ferndale, MI (US)

(73) Assignees: Wayne State University, Detroit, MI (US); Wayne State University, Detroit, MI (US)

(21) Appl. No.: 17/636,107

(22) PCT Filed: Aug. 19, 2020
(86) PCT No.: PCT/US2020/047016

§ 371 Date: Feb. 17, 2022

Related U.S. Application Data
(60) Provisional application No. 62/888,747, filed on Aug. 19, 2019.

(51) Int. Cl. A61B 6/03; A61K 51/10; A61K 38/19; A61K 31/164; A61P 35/00; A61P 39/04; C07K 16/24

(52) U.S. Cl. CPC A61B 6/037; A61K 51/1021; A61K 51/1093; A61K 38/193; A61K 31/164; A61P 35/00; A61P 39/04; C07K 16/244; A61B 6/4417

(57) ABSTRACT

Compositions and methods for in vivo immunoimaging IL-12 as a marker of IL-12—producing activated antigen presenting cells (APCs) in a subject are provided according to aspects of the present disclosure which include: administering a labeled-antibody conjugate to a subject, wherein the labeled-antibody conjugate includes 1) an antibody or antibody fragment that specifically binds to IL-12, and 2) a detection label conjugated to the antibody or antibody fragment, wherein the detection label is a radionuclide tracer, fluorophore, or nanoparticle, and wherein the labeled-antibody conjugate specifically binds to IL-12; and detecting the presence of the labeled-antibody conjugate in the subject in vivo by imaging. According to embodiments of the present disclosure, the subject is human and the antibody or antibody fragment specifically binds to human IL-12.

### REFERENCE TO RELATED APPLICATION

This application claims priority from U.S. Provisional Patent Application Ser. No. 62/888,747, filed Aug. 19, 2019, the entire content of which is incorporated herein by reference.

### GRANT REFERENCE

This invention was made with government support under Grant No. R37 CA220482, awarded by the National Institutes of Health. The Government has certain rights in the invention.

### FIELD OF THE INVENTION

The present disclosure generally relates to antibody conjugates, to methods for imaging antibody conjugates in vivo, and methods and compositions relating to antibody conjugates. The present disclosure specifically relates to antibody conjugates which specifically recognize interleukin 12 (IL-12), to methods for imaging IL-12 antibody conjugates in vivo, methods for in vivo immunoimaging of IL-12-producing activated antigen presenting cells (APCs), methods for identifying and localizing active inflammation and/or infection, and methods for assessing an effect of an immune-mediated treatment for cancer, as well as assessment of treatments for injury, inflammatory conditions, autoimmune conditions, and infection.

### BACKGROUND OF THE INVENTION

Innate immune cells guide adaptive immune cell differentiation and behavior by producing specific cytokines. Interleukin-12 (IL-12) is a critical cytokine produced by dendritic cells (DCs) and other immune cells, necessary for the induction of interferon-γ (IFN-γ)-producing Th1 subset CD8+ and CD4+ T cells, both of which are beneficial in an anti-tumor response. IL-12 further plays a central role in pro-inflammatory responses and conditions. Interleukin 12 (IL-12) production by IL-12-producing activated antigen presenting cells (APCs) skews CD4 T cells toward the inflammatory Th1 subtype, and promotes CD8 T cell development and behavior. The effects of IL-12, and its downstream promotion of both Th1 and CD8 T cells have been implicated in anti-tumor immunity, tissue-damaging autoimmunity, and infectious disease.

Of the total T cell population, infiltrating CD8⁺ cytotoxic T lymphocytes (CTL) are the most important subset for anti-tumor immunity, as they directly lyse their target tumor cells. Prior to CTL activation, multiple signaling cascades occur involving the release of inflammatory cytokines from APCs which trigger activation and guide maturation outcomes of both CTL and CD4⁺ T helper (Th) cells. The production of IL-12 by activated APCs during immune priming promotes effector function in CTL and differentiates naïve CD4⁺ T cells toward the interferon-γ (IFN-γ)-producing Th1 subset, which further supports CTL activity, resulting in stronger anti-tumor immunity. These same signaling pathways are critical for inflammatory T cell activation in various forms of autoimmunity and response to infectious disease.

Recent emerging tumor-targeted immunotherapy strategies have met with positive and durable outcomes in the clinic, and yet at least half of cancer patients remain non-responsive thereby creating urgency to monitor and gauge an immunotherapy's success in a timely manner. Multiple sequential biopsies post-treatment are not always ethically feasible and only represent a small region of a heterogeneous tumor.

There is an ongoing need for tools and methods to monitor and gauge therapeutic success of an immunotherapy in situ. Image-guided focal analysis of intratumoral immune activity according to aspects of the present disclosure, provides non-invasive, real-time efficacy predictions to aid in assessment of the effectiveness of an immunotherapy.

In vivo monitoring of IL-12 production via imaging according to aspects of the present disclosure provides a predictive measure of subsequent adaptive immune activation in conditions involving immune activation in a subject including tumor immunology, injury, inflammatory conditions, autoimmune conditions, and infection.

### SUMMARY OF THE INVENTION

Methods for in vivo immunoimaging IL-12 as a marker of IL-12-producing activated antigen presenting cells (APCs) in a subject are provided according to aspects of the present disclosure which include: administering a labeled-antibody conjugate to a subject, wherein the labeled-antibody conjugate includes 1) an antibody or antibody fragment that specifically binds to IL-12, and 2) a detection label conjugated to the antibody or antibody fragment, wherein the detection label is or includes a radionuclide tracer, a fluorophore, a nanoparticle, or any two or more thereof, and wherein the labeled-antibody conjugate specifically binds to IL-12; and detecting the presence of the labeled-antibody conjugate in the subject in vivo by imaging. According to embodiments of the present disclosure, the subject is human and the antibody or antibody fragment specifically binds to human IL-12.

According to embodiments of the present disclosure, methods for in vivo immunoimaging IL-12 as a marker of IL-12-producing activated antigen presenting cells (APCs) in a subject are provided which include: administering a labeled-antibody conjugate to a subject wherein the subject has a condition selected from the group consisting of: cancer, an inflammatory condition, an autoimmune condition, an infection, an injury, or a combination of any two or more thereof, wherein the labeled-antibody conjugate includes 1) an antibody or antibody fragment that specifically binds to IL-12, and 2) a detection label conjugated to the antibody or antibody fragment, wherein the detection label is or includes a radionuclide tracer, a fluorophore, a nanoparticle, or any two or more thereof, and wherein the labeled-antibody conjugate specifically binds to IL-12; and detecting the presence of the labeled-antibody conjugate in the subject in vivo by imaging. According to embodiments of the present disclosure, the subject is human and the antibody specifically binds to human IL-12.

According to embodiments of the present disclosure, methods for in vivo immunoimaging IL-12 as a marker of IL-12—producing activated antigen presenting cells (APCs) in a subject are provided which include: administering a labeled-antibody conjugate to a subject wherein the subject has a condition selected from the group consisting of: cancer, an inflammatory condition, an autoimmune condition, an infection, an injury, or a combination of any two or more thereof, wherein the labeled-antibody conjugate includes 1) an antibody or antibody fragment that specifically binds to IL-12, and 2) a detection label conjugated to the antibody or antibody fragment, wherein the detection label is or includes a radionuclide tracer, a fluorophore, a nanoparticle, or any two or more thereof, and wherein the labeled-antibody conjugate specifically binds to IL-12; and detecting the presence of the labeled-antibody conjugate in the subject in vivo by imaging, wherein the subject received an immunotherapy prior to administering and detecting the presence of the labeled-antibody conjugate, and wherein the mechanism of action of the immunotherapy results in an increased number of IL-12—producing activated APCs in the subject; or wherein administering and detecting the presence of the labeled-antibody conjugate is performed prior to administration of a therapy to determine the state of active immunity in the subject. According to embodiments of the present disclosure, the subject is human and the antibody or antibody fragment specifically binds to human IL-12.

According to embodiments of the present disclosure, the immunotherapy is an immune checkpoint inhibitor, a receptor agonist, a cytokine, a vaccine, an adoptive cell transfer therapy, an oncolytic virus, or any therapeutic wherein the mechanism of action of the therapeutic is an increased number of tumor-infiltrating lymphocytes in the subject and/or an increased activation state of a tumor-infiltrating lymphocyte population in the subject. According to embodiments, the immune checkpoint inhibitor is an inhibitor of a negative regulatory signaling pathway including PD-1, PD-L1, CTLA-4, TIM-3, or LAG-3.

According to embodiments, the immune checkpoint inhibitor selected from atezolizumab, avelumab, durvalumab, ipilimumab, nivolumab, pembrolizumab, and an antigen-binding fragment of any one of the foregoing.

According to embodiments, the receptor agonists are antibodies or other molecules inducing activation of stimulatory receptors including 4-1BB, CD27, CD40, GITR, ICOS, or OX40.

According to embodiments, cytokine therapies may include IL-2, IFN-α, or IL-15.

Vaccine platforms may include protein, peptide, recombinant vectors such as viruses or DNA, whole tumor cells with or without engineered immune stimulatory modifications, or dendritic cell vaccines.

Adoptive cell therapies include chimeric antigen receptor (CAR) T cells, expanded tumor infiltrating cells, and T cells engineered to express specific T cell receptors.

Oncolytic viral therapies include the engineered herpes simplex virus type I encoding granulocyte-macrophage colony-stimulating factor (GM-CSF) talimogene laherparepvec (T-VEC).

According to embodiments of the present disclosure, the antibody or antibody fragment that specifically binds to IL-12 and which is included in a labeled-antibody conjugate that specifically binds to IL-12 is selected from: a monoclonal antibody, an antibody fragment, or combination thereof.

According to embodiments of the present disclosure, the antibody that specifically binds to IL-12 and which is included in a labeled-antibody conjugate that specifically binds to IL-12 is an Fab fragment.

According to embodiments of the present disclosure, the antibody that specifically binds to IL-12 and which is included in a labeled-antibody conjugate that specifically binds to IL-12 is a diabody.

According to embodiments of the present disclosure, the antibody that specifically binds to IL-12 and which is included in a labeled-antibody conjugate that specifically binds to IL-12 is an Fab′2 antibody fragment, a minibody, an ScFv antibody fragment, or a nanobody.

According to embodiments of the present disclosure, the radionuclide tracer included in a labeled-antibody conjugate that specifically binds to IL-12 is conjugated to the anti-IL-12 antibody or anti-IL-12 antibody fragment via a bifunctional chelator or linker, wherein the bifunctional chelator or linker is attached to the anti-IL-12 antibody and the radionuclide tracer.

According to embodiments of the present disclosure, the radionuclide tracer included in a labeled-antibody conjugate that specifically binds to IL-12 is conjugated to the anti-IL-12 antibody or anti-IL-12 antibody fragment via a bifunctional chelator, wherein the bifunctional chelator is selected from:
2,2′,2″-(10-(2-((2-(2,5-dioxo-2,5-dihydro-1H-pyrrol-1-yl)ethyl)amino)-2-oxoethyl)-1,4,7,10-tetraazacyclododecane-1,4,7-triyl)triacetic acid (DOTA-maleimide); 2,2′,2″-(10-(2-((2,5-dioxopyrrolidin-1-yl)oxy)-2-oxoethyl)-1,4,7,10-tetraazacyclododecane-1,4,7-triyl) triacetic acid (DOTA-NHS); S-2-(4-Aminobenzyl)-1,4,7,10-tetraazacyclododecane tetraacetic acid (p-NH2-Bn-DOTA); S-2-(4-Isothiocyanatobenzyl)-1,4,7,10-tetraazacyclododecane tetraacetic acid (p-SCN-Bn-DOTA); 1,4,7,10-Tetraazacyclododecane-1,4,7-tris(acetic acid)-10-(azidopropyl ethylacetamide) (Azido-mono-amide-DOTA); 2,2′,2″-(10-(1-carboxy-4-((4-isothiocyanatobenzyl)amino)-4-oxobutyl)-1,4,7,10-tetraazacyclododecane-1,4,7-triyl)triacetic acid (p-NCS-Bz-DOTA-GA); 2,2′,2″-(10-(1-carboxy-4-((2-(2,5-dioxo-2,5-dihydro-1H-pyrrol-1-yl)ethyl)amino)-4-oxobutyl)-1,4,7,10-tetraazacyclododecane-1,4,7-triyl)triacetic acid (Maleimide-DOTA-GA); 2,2′,2″-(10-(4-((2-((((1R,8S,9s)-bicyclo[6.1.0]non-4-yn-9-ylmethoxy)carbonyl)amino)ethyl)amino)-1-carboxy-4-oxobutyl)-1,4,7,10-tetraazacyclododecane-1,4,7-triyl)triacetic acid (BCN-DOTA-GA); S-2-(4-Aminobenzyl)-diethylenetriamine pentaacetic acid (p-NH2-Bn-DTPA); S-2-(4-Isothiocyanatobenzyl)-diethylenetriamine pentaacetic acid (p-SCN-Bn-DTPA); [(R)-2-Amino-3-(4-isothiocyanatophenyl)propyl]-trans-(S,S)-cyclohexane-1,2-diamine-pentaacetic acid (p-SCN-Bn-CH-A″-DTPA); 2,2′-(1-carboxy-2-(carboxymethyl)-13-(2,5-dioxo-2,5-dihydro-1H-pyrrol-1-yl)-10-oxo-2,5,8,11-tetraazatridecane-5,8-diyl)diacetic acid (Maleimide-DTPA); 2,2′-(7-(2-((2,5-dioxopyrrolidin-1-yl)oxy)-2-oxoethyl)-1,4,7-triazonane-1,4-diyl)diacetic acid (NOTA-NHS); 2,2′-(7-(2-((2-(2,5-dioxo-2,5-dihydro-1H-pyrrol-1-yl)ethyl)amino)-2-oxoethyl)-1,4,7-triazonane-1,4-diyl)diacetic acid (Maleimide-NOTA); 2-S-(4-Isothiocyanatobenzyl)-1,4,7-triazacyclononane-1,4,7-triacetic acid (p-SCN-Bn-NOTA); 2,2′-(7-(1-carboxy-4-((2,5-dioxopyrrolidin-1-yl)oxy)-4-oxobutyl)-1,4,7-triazonane-1,4-diyl)diacetic acid (NODA-GA-NHS); 2,2′-(7-(1-carboxy-4-((2-(2,5-dioxo-2,5-dihydro-1H-pyrrol-1-yl)ethyl)amino)-4-oxobutyl)-1,4,7-triazonane-1,4-diyl)diacetic acid (Maleimide-NODA-GA); 2,2′-(7-(1-carboxy-4-((4-isothiocyanatobenzyl)amino)-4-oxobutyl)-1,4,7-triazonane-1,4-diyl)diacetic acid (p-NCS-benzyl-NODA-GA); 2,2′-(7-(4-isothiocyanatobenzyl)-1,4,7-triazonane-1,4-diyl) diacetic acid (NCS-MP-NODA); 2,2′-(7-(4-((2-((((1R,8S,9s)-bicyclo[6.1.0]non-4-yn-9-ylmethoxy)carbonyl)amino)ethyl)amino)-1-carboxy-4-oxobutyl)-1,4,7-triazonane-1,4-diyl)diacetic acid (BCN-NODA-GA); 1,4,7-Triazacyclononane-1,4-bis(acetic acid)-7-(3-azidopropylacetamide) (NO2A-Azide); 3,6,9,15-Tetraazabicyclo[9.3.1] pentadeca-1(15),11,13-triene-4-S-(4-aminobenzyl)-3,6,9-triacetic acid (p-NH2-Bn-PCTA); 3,6,9,15-Tetraazabicyclo [9.3.1]pentadeca-1(15),11,13-triene-4-S-(4-isothiocyanatobenzyl)-3,6,9-triacetic acid (p-SCN-BN-PCTA); 1-(4-isothiocyanatophenyl)-3-[6,17-dihydroxy-7,10,18,21-tetraoxo-27-(N-acetylhydroxylamino)-6,11,17,22-tetraazaheptaeicosine] thiourea (p-SCN-Bn-DFO); and N-(3,11,14,22,25,33-hexaoxo-4,10,15,21,26,32-hexaaza-10,21,32-trihydroxytetratriacontane)maleimide (Maleimide-DFO);

or a combination of any two or more thereof.

According to embodiments of the present disclosure, the radionuclide tracer included in a labeled-antibody conjugate that specifically binds to IL-12 is conjugated to the anti-IL-12 antibody or anti-IL-12 antibody fragment via a bifunctional chelator wherein the bifunctional chelator is p-SCN-Bn-DFO.

According to embodiments of the present disclosure, the radionuclide tracer included in a labeled-antibody conjugate that specifically binds to IL-12 is selected from: ¹¹C, ¹³N, ¹⁵O, ¹⁸F, ⁴⁴Sc, ⁴⁵Ti, ⁵²Mn, ⁶⁴Cu, ⁶⁸Ga, ⁴⁴Sc, ⁷⁶Br, ⁸²Rb, ⁸⁶Y, ⁸⁹Zr, ⁹⁰Y, ^{99m}Tc, ¹¹¹In, ¹²⁴I, ¹³¹I, ⁴³K, ⁵²Fe, ⁵⁷Co, ⁶⁷Cu, ⁶⁷Ga, ⁷⁷Br, ⁸¹Rb, ^{81m}Kr, ^{87m}Sr, ⁸⁹Zr, ^{113m}In, ¹²³I, ¹²⁵I, ¹²⁷Cs ¹²⁹Cs, ¹³²I, ¹⁷⁷Lu, ¹⁸⁶Re, ¹⁹⁷Hg, ²⁰³Pb, ²⁰⁶Bi, ⁸²Sr, ¹⁸⁸Re, ⁶⁰Cu, ⁶¹Cu, ⁶²Cu, ²²⁵Ac, ²²⁵Ra, and a combination of any two or more thereof.

According to embodiments of the present disclosure, the radionuclide tracer included in a labeled-antibody conjugate that specifically binds to IL-12 is ⁸⁹Zr or ¹⁸F.

According to embodiments of the present disclosure, detecting the presence of the labeled-antibody conjugate in the subject in vivo includes positron emission tomography (PET) imaging, single photon emission computed tomography (SPECT) imaging, or both PET and SPECT.

According to embodiments of the present disclosure, detecting the presence of the labeled-antibody conjugate in the subject in vivo includes positron emission tomography (PET) imaging.

According to embodiments of the present disclosure, the labeled-antibody conjugate includes a fluorophore attached to the anti-IL-12 antibody or anti-IL-12 antibody fragment and detecting the presence of the labeled-antibody conjugate in the subject in vivo includes optical imaging.

According to embodiments of the present disclosure, the labeled-antibody conjugate includes a nanoparticle attached to the anti-IL-12 antibody or anti-IL-12 antibody fragment and detecting the presence of the labeled-antibody conjugate in the subject in vivo includes magnetic resonance imaging (MRI) and/or magnetic particle imaging (MPI).

According to embodiments of the present disclosure, specific binding of the labeled-antibody conjugate to IL-12 indicates the presence of IL-12—producing activated APCs.

According to embodiments of the present disclosure, the presence of the labeled-antibody conjugate is detected in the subject in real time.

Labeled-antibody conjugates are provided according to embodiments of the present disclosure which include 1) an antibody or antibody fragment that specifically binds to IL-12, and 2) a detection label conjugated to the antibody or antibody fragment, wherein the labeled-antibody conjugate specifically binds to IL-12. According to embodiments, pharmaceutical compositions including the labeled-antibody conjugate and a pharmaceutically acceptable carrier are provided. According to embodiments of the present disclosure, the antibody or antibody fragment specifically binds to human IL-12. According to embodiments of the present disclosure, the antibody or antibody fragment specifically binds to mouse IL-12.

Labeled-antibody conjugates are provided according to embodiments of the present disclosure which include 1) an antibody or antibody fragment that specifically binds to IL-12, and 2) a detection label conjugated to the antibody or antibody fragment, wherein the detection label is or includes a radionuclide tracer, a fluorophore, a nanoparticle, or any two or more thereof, and wherein the labeled-antibody conjugate specifically binds to IL-12. According to embodiments, pharmaceutical compositions including the labeled-antibody conjugate and a pharmaceutically acceptable carrier are provided. According to embodiments of the present disclosure, the antibody or antibody fragment specifically binds to human IL-12. According to embodiments of the present disclosure, the antibody or antibody fragment specifically binds to mouse IL-12.

Labeled-antibody conjugates are provided according to embodiments of the present disclosure which include 1) an antibody or antibody fragment that specifically binds to IL-12, and 2) a detection label conjugated to the antibody or antibody fragment, wherein the detection label is a radionuclide tracer, and wherein the labeled-antibody conjugate specifically binds to IL-12. According to embodiments, pharmaceutical compositions including a labeled-antibody conjugate and a pharmaceutically acceptable carrier are provided. According to embodiments of the present disclosure, the antibody or antibody fragment specifically binds to human IL-12. According to embodiments of the present disclosure, the antibody or antibody fragment specifically binds to mouse IL-12.

Labeled-antibody conjugates are provided according to embodiments of the present disclosure which include 1) a monoclonal antibody or monoclonal antibody fragment that specifically binds to IL-12, and 2) a detection label conjugated to the monoclonal antibody or monoclonal antibody fragment, wherein the detection label is or includes a radionuclide tracer, a fluorophore, a nanoparticle, or any two or more thereof, and wherein the labeled-antibody conjugate specifically binds to IL-12.

Labeled-antibody conjugates are provided according to embodiments of the present disclosure which include 1) an Fab fragment of an antibody that specifically binds to IL-12, and 2) a detection label conjugated to the Fab fragment, wherein the detection label is or includes a radionuclide tracer, a fluorophore, a nanoparticle, or any two or more thereof and wherein the labeled-antibody conjugate specifically binds to IL-12.

Labeled-antibody conjugates are provided according to embodiments of the present disclosure which include 1) an Fab′2 antibody fragment, a minibody, an ScFv antibody fragment, or a nanobody that specifically binds to IL-12, and 2) a detection label conjugated to the an Fab′2 antibody fragment, a minibody, an ScFv antibody fragment, or a nanobody, wherein the detection label is or includes a radionuclide tracer, a fluorophore, a nanoparticle, or any two or more thereof, and wherein the labeled-antibody conjugate specifically binds to IL-12.

Labeled-antibody conjugates are provided according to embodiments of the present disclosure which include 1) a diabody that specifically binds to IL-12, and 2) a detection label conjugated to the diabody, wherein the detection label is or includes a radionuclide tracer, a fluorophore, a nanoparticle, or any two or more thereof, and wherein the labeled-antibody conjugate specifically binds to IL-12.

Labeled-antibody conjugates are provided according to embodiments of the present disclosure wherein the radionuclide tracer is conjugated to the antibody or antibody fragment with a bifunctional chelator or linker, and wherein the bifunctional chelator or linker is attached to the antibody or antibody fragment and to the radionuclide tracer. According to embodiments of the present disclosure the bifunctional chelator includes a chelator selected from: 1,4,7-Triazacyclononane (TACN); 1,4,7,10-Tetraazacyclododecane (Cyclen); 1,4,7,10-Tetraazacyclododecane-1,7-diacetic acid (DO2A); 1,4,7,10-Tetraazacyclododecane-1,4,7-triacetic acid trisodium salt (DO3A); 1,4,7,10-Tetraazacyclododecane-1,4,7,10-tetraacetic acid (DOTA); 1,4,7,10-Tetraazacyclododecane-1,4,7,10-tetra(methylene phosphonic acid) (DOTP); 1,4,8,11-Tetraazacyclotetradecane-1,4,8,11-tetraacetic acid (TETA); 2,2′,2″,2′″-(1,4,8,11-tetraazacyclotetradecane-1,4,8,11-tetrayl)tetraacetamide (TETAM); 1,4,7,10-Tetrakis (carbamoylmethyl)-1,4,7,10-tetraazacyclododecane (DOTAM); 2,2′,2″-(1,4,7,10-tetraazacyclododecane-1,4,7-triyl)triacetamide (DO3AM); 1,8-Diamino-3,6,10,13,16,19-hexaazabicyclo [6,6,6]-eicosane (DiAmSar); 1,4,8,11-Tetraazabicyclo[6.6.2]hexadecane (CB-Cyclam); 2,2′-(1,4,8,11-tetraazabicyclo[6.6.2]hexadecane-4,11-diyl)diacetic acid (CB-TE2A); 1,4,7-Triazacyclononane-1,4,7-triacetic acid (NOTA); 1,4,7-Triazacyclononane-1,4,7-tri(methylene phosphonic acid) (NOTP); 3-(((4,7-bis ((hydroxy(hydroxymethyl)phosphoryl)methyl)-1,4,7-triazonan-1-yl)methyl)(hydroxy)phosphoryl)propanoic acid (NOPO); 2,2′,2″-(1,4,7-triazacyclononane-1,4,7-triyl)triacetamide (NOTAM); 2,2′,2″,2′″-((((carboxymethyl)azanediyl)bis(ethane-2,1-diyl))bis(azanetriyl))tetraacetic acid (DTPA); 3,6,9,15-Tetraazabicyclo[9.3.1]pentadeca-1(15),11,13-triene-3,6,9-triacetic acid (PCTA); 2,2′,2″,2′″-(1,4,7,10-tetraazacyclotridecane-1,4,7,10-tetrayl)tetraacetic acid (TRITA); 2,2′,2″,2′″-(1,4,7,10-tetraazacyclotridecane-1,4,7,10-tetrayl)tetraacetamide (TRITAM); 2,2′,2″-(1,4,7,10-tetraazacyclotridecane-1,4,7-triyl)triacetamide (TRITRAM); and 3,3′,3″-(((1,4,7-triazonane-1,4,7-triyl) tris(methylene))tris(hydroxyphosphoryl))tripropanoic acid (TRAP) or a combination of any two or more thereof.

According to embodiments of the present disclosure, the bifunctional chelator is p-SCN-Bn-DFO.

According to embodiments of the present disclosure, a labeled-antibody conjugate includes a radionuclide tracer selected from: ¹¹C, ¹³N, ¹⁵O, ¹⁸F, ⁴⁴Sc, ⁴⁵Ti, ⁵²Mn, ⁶⁴Cu, ⁶⁸Ga, ⁴⁴Sc, ⁷⁶Br, ⁸²Rb, ⁸⁶Y, ⁸⁹Zr, ⁹⁰Y, ^{99m}Tc, ¹¹¹In, ¹²⁴I, ¹³¹I, ⁴³K, ⁵²Fe, ⁵⁷Co, ⁶⁷Cu, ⁶⁷Ga, ⁷⁷Br, ⁸¹Rb, ^{81m}Kr, ^{87m}Sr, ⁸⁹Zr, ^{113m}In, ¹²³I, ¹²⁵I, ¹²⁷Cs, ¹²⁹Cs, ¹³²I, ¹⁷⁷Lu, ¹⁸⁶Re, ¹⁹⁷Hg, ²⁰³Pb, ²⁰⁶Bi, ⁸²Sr, ¹⁸⁸Re, ⁶⁰Cu, ⁶¹Cu, ⁶²Cu, ²²⁵Ac, ²²⁵Ra, and a combination of any two or more thereof.

According to embodiments of the present disclosure, a labeled-antibody conjugate includes a radionuclide tracer selected from: ⁸⁹Zr, ¹⁸F, or a combination thereof.

### BRIEF DESCRIPTION OF THE DRAWINGS

FIG. 1 is a schematic diagram showing an imaging agent for IL-12 according to aspects of the present disclosure.

FIG. 2 is a schematic diagram showing an intact engineered antibody (1) and antibody fragments: a minibody (2), diabody or Fab (3), scFv (4) and a nanobody (5).

FIGS. 3A and 3B show results of IL-12 PET imaging of tumors treated with an oncolytic adenovirus encoding the dendritic cell maturation cytokine granulocyte macrophage-colony stimulating factor (Adv/GM-CSF).

FIG. 3A is a graph showing results of analysis of [⁸⁹Zr]Zr-IgG (non-specific control) and [⁸⁹Zr]Zr-anti-IL12 uptake which demonstrate a statistically significant difference in treated (Tx) tumors imaged via IL-12 PET compared to untreated control (UTx) tumors. Uptake of the irrelevant IgG ([⁸⁹Zr]Zr-IgG) displayed a lower accumulation for both cohorts. Importantly, lower accumulation of [⁸⁹Zr]Zr-IgG was observed when compared with [⁸⁹Zr]Zr-anti-IL12 in Tx groups.

FIG. 3B is a graph showing that results of qRT-PCR of IL-12b in snap-frozen tissue sections validated the [⁸⁹Zr]Zr-anti-IL12 uptake, demonstrating a higher level of IL-12b mRNA transcripts in the Tx group compared to UTx.

FIGS. 4A, 4B, 4C, 4D, and 4E show results of PET following [⁸⁹Zr]Zr-anti-IL12 administration in a lipopolysaccharide (LPS)-induced inflammation model.

FIG. 4A shows two images demonstrating that [⁸⁹Zr]Zr-anti-IL-12 had higher accumulation on the site of LPS injection compared to contralateral (C.L.) muscle in the same animal and compared to control untreated mice.

FIG. 4B is a graph showing a time activity curve that displays increased [⁸⁹Zr]Zr-anti-IL-12 accumulation in the LPS-injected muscle versus C.L. muscle at 24-72 h post-injection.

FIG. 4C is a graph showing tissue distribution and illustrating the pharmacokinetics of the [⁸⁹Zr]Zr-anti-IL-12 at 24 h post-injection (p.i.).

FIG. 4D is a graph showing that the inflamed site (“with LPS”) has higher accumulation of [⁸⁹Zr]Zr-anti-IL-12 compared to the C.L. muscle and compared to control muscle tissues (“no LPS”) excised for biodistribution studies.

FIG. 4E is a graph showing that inguinal lymph nodes had higher uptake of [⁸⁹Zr]Zr-anti-IL-12 in the LPS-treated groups compared to control, untreated mice.

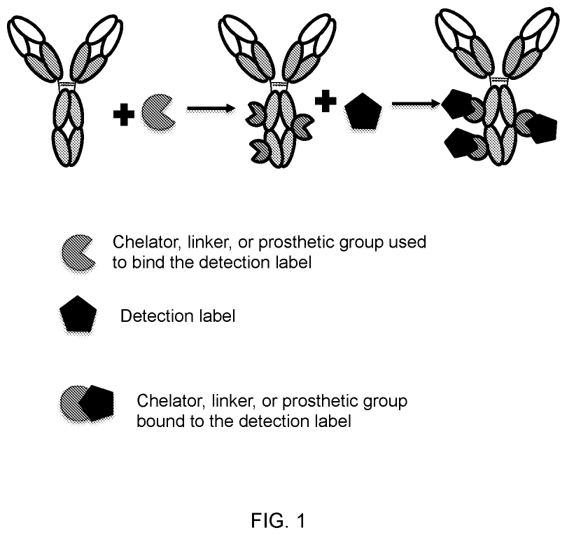

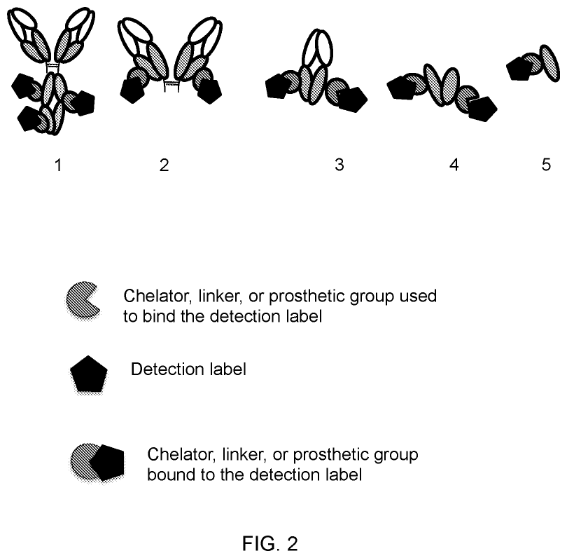

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; G. T. Hermanson, Bioconjugate Techniques, 2nd Edition, Academic Press, 2008; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

While the following terms are believed to be well understood by one of ordinary skill in the art, definitions are set forth to facilitate explanation of the presently-disclosed subject matter.

The term “detection label” refers to a chemical moiety that can be covalently attached to an antibody and that functions to provide a detectable signal. Examples of such labels include fluorescent moieties, chemiluminescent moieties, bioluminescent moieties, nanoparticles, magnetic particles, metal-containing particles, and radiolabels, such as radionuclide tracers.

The term “nanoparticle” as used herein refers to a particle having a size parameter measured on a nanometer scale, i.e. below 1 micron, and typically below 500 nm. For example, a nanoparticle has a longest dimension below 1 micron in length, such as 1 nm to 500 nm, 5 nm to 100 nm, 10 nm to 90 nm, 20 nm to 80 nm, 30 nm to 75 nm, 40 nm to 70 nm, 50 nm to 60 nm, such as a longest dimension of about 1 nm, 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, or 100 nm.

The nanoparticles can have any shape, such as spheres, rods, cubes, wires, plates, irregular, or mixed shapes.

The nanoparticles can be made of a synthetic or natural material, or combination thereof, which is directly imaged, or may themselves include a detection label.

The nanoparticles may contain, or consist of, a metal, such as but not limited to, gold, iron, zinc, silver, copper, cobalt, cadmium, nickel, gadolinium, chromium, tin, aluminum, palladium, manganese, titanium, an oxide of any thereof, alloys or mixtures of any two or more thereof.

The particles may be, or include, carbon, such as carbon nanotubes.

The term “antibody” is used herein in its broadest sense and specifically refers to monoclonal antibodies, polyclonal antibodies, dimers, multimers, multispecific antibodies (e.g., bispecific antibodies), and antibody fragments, so long as they exhibit the desired biological activity (see e.g., Miller et al. (2003) J Immunology 170:4854-4861). Antibodies may be murine, human, humanized, chimeric, or derived from other species. An antibody is a protein that is capable of recognizing and binding to a specific antigen, such as, e.g., IL-12. (See e.g., Janeway, C., Travers, P., Walport, M., Shlomchik (2001) Immuno Biology, 5th Ed., Garland Publishing, New York). A target antigen may have numerous binding sites, also called epitopes, recognized by complementarity determining regions of antibodies. In embodiments, each antibody that specifically binds to a different epitope has a different structure. Thus, one antigen may have more than one corresponding antibody. An antibody includes a full-length immunoglobulin molecule or an immunologically-active portion of a full-length immunoglobulin molecule, such as, e.g., a molecule that contains an antigen binding site that immunospecifically recognizes and binds to an antigen of a target of interest or part thereof, such as, e.g., IL-12. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In some embodiments, however, the immunoglobulin is of rodent or human origin. According to embodiments, the antibody is an antibody fragment and in specific embodiments, the antibody fragment is a diabody.

The term “specifically recognizes and binds to” refers to antibodies that are capable of binding to an antigen of interest, such as, e.g., IL-12, with sufficient binding affinity such that the antibody is useful in targeting the antigen of interest. As an example, the antibodies may have a binding affinity K_{D} of from about 10⁻⁴ to about 10⁻¹⁵, or from about 10⁻⁶ to about 10⁻¹³, or from about 10⁻⁷ to about 10⁻¹², or from about 10⁻⁹ to about 10⁻¹⁰, for the antigen of interest. As another example, where the antibody is one that binds to IL-12, it will preferentially bind to IL-12 as opposed to other antigens and/or extracellular components. As a further example, where the antibody is one that binds to IL-12, it may not significantly cross-react with other, non-IL-12, antigens. In embodiments, the extent of binding of the antibody to non-IL-12 antigens, and/or other materials, is less than about 10% (such as, e.g., 0% to about 9%), as determined by standard techniques known to those of ordinary skill in the art, such as, e.g., by flow cytometric analysis.

The terms “interleukin 12” and “IL-12” are synonyms that refer to a cytokine. IL-12 may refer to IL-12 of any species including both human and mouse IL-12.

Human interleukin-12 (IL-12, p70) is a 70 kDa protein which includes two disulfide-linked subunits, p35 and p40. IL-12 is produced by activated antigen presenting cells, including macrophages, monocytes, dendritic cells, and neutrophils, as well as some cell lines. Structural characteristics of IL-12 are well-known, including amino acid sequence, nucleic acids encoding the protein, and crystal structure, see for example C. Yoon et al., EMBO J., 19(14):3530-3541, 2000.

The term “bifunctional chelator” refers to a chemical moiety that attaches an antibody to a radionuclide label. Bifunctional chelators include 1) a chelator moiety functional to bind a radionuclide label and 2) a reactive functional group. Bifunctional chelators function by complexing a radionuclide with the chelator moiety and by covalently attaching the chelator moiety (and complexed radionuclide) to an antibody via reaction of the reactive functional group with a corresponding reactive functional group of the antibody.

For example, bifunctional chelators may be covalently attached to a primary amine group, a hydroxyl group, and/or a cysteine amino acid of an antibody. Examples of suitable chelator moieties include: 1,4,7-Triazacyclononane (TACN); 1,4,7,10-Tetraazacyclododecane (Cyclen); 1,4,7,10-Tetraazacyclododecane-1,7-diacetic acid (DO2A); 1,4,7,10-Tetraazacyclododecane-1,4,7-triacetic acid trisodium salt (DO3A); 1,4,7,10-Tetraazacyclododecane-1,4,7,10-tetraacetic acid (DOTA); 1,4,7,10-Tetraazacyclododecane-1,4,7,10-tetra(methylene phosphonic acid) (DOTP); 1,4,8,11-Tetraazacyclotetradecane-1,4,8,11-tetraacetic acid (TETA); 2,2′,2″,2′″-(1,4,8,11-tetraazacyclotetradecane-1,4,8,11-tetrayl)tetraacetamide (TETAM); 1,4,7,10-Tetrakis (carbamoylmethyl)-1,4,7,10-tetraazacyclododecane (DOTAM); 2,2′,2″-(1,4,7,10-tetraazacyclododecane-1,4,7-triyl)triacetamide (DO3AM); 1,8-Diamino-3,6,10,13,16,19-hexaazabicyclo [6,6,6]-eicosane (DiAmSar); 1,4,8,11-Tetraazabicyclo[6.6.2]hexadecane (CB-Cyclam); 2,2′-(1,4,8,11-tetraazabicyclo[6.6.2]hexadecane-4,11-diyl)diacetic acid (CB-TE2A); 1,4,7-Triazacyclononane-1,4,7-triacetic acid (NOTA); 1,4,7-Triazacyclononane-1,4,7-tri(methylene phosphonic acid) (NOTP); 3-(((4,7-bis ((hydroxy(hydroxymethyl)phosphoryl)methyl)-1,4,7-triazonan-1-yl) methyl)(hydroxy)phosphoryl)propanoic acid (NOPO); 2,2′,2″-(1,4,7-triazacyclononane-1,4,7-triyl)triacetamide (NOTAM); 2,2′,2″,2′″-((((carboxymethyl)azanediyl)bis(ethane-2,1-diyl))bis(azanetriyl))tetraacetic acid (DTPA); 3,6,9,15-Tetraazabicyclo[9.3.1]pentadeca-1(15),11,13-triene-3,6,9-triacetic acid (PCTA); 2,2′,2″,2′″-(1,4,7,10-tetraazacyclotridecane-1,4,7,10-tetrayl)tetraacetic acid (TRITA); 2,2′,2″,2′″-(1,4,7,10-tetraazacyclotridecane-1,4,7,10-tetrayl)tetraacetamide (TRITAM); 2,2′,2″-(1,4,7,10-tetraazacyclotridecane-1,4,7-triyl)triacetamide (TRITRAM); and 3,3′,3″-(((1,4,7-triazonane-1,4,7-triyl) tris(methylene))tris(hydroxyphosphoryl))tripropanoic acid (TRAP); or a combination of any two or more thereof.

Examples of suitable reactive functional groups capable of attaching a chelator moiety to a primary amine group, a hydroxyl group, and/or a cysteine amino acid of an antibody are known to those of skill in the art (see e.g., Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50).

Examples of suitable bifunctional chelators include 2,2′,2″-(10-(2-((2-(2,5-dioxo-2,5-dihydro-1H-pyrrol-1-yl)ethyl)amino)-2-oxoethyl)-1,4,7,10-tetraazacyclododecane-1,4,7-triyl)triacetic acid (DOTA-maleimide); 2,2′,2″-(10-(2-((2,5-dioxopyrrolidin-1-yl)oxy)-2-oxoethyl)-1,4,7,10-tetraazacyclododecane-1,4,7-triyl) triacetic acid (DOTA-NHS); S-2-(4-Aminobenzyl)-1,4,7,10-tetraazacyclododecane tetraacetic acid (p-NH2-Bn-DOTA); S-2-(4-Isothiocyanatobenzyl)-1,4,7,10-tetraazacyclododecane tetraacetic acid (p-SCN-Bn-DOTA); 1,4,7,10-Tetraazacyclododecane-1,4,7-tris(acetic acid)-10-(azidopropyl ethylacetamide) (Azido-mono-amide-DOTA); 2,2′,2″-(10-(1-carboxy-4-((4-isothiocyanatobenzyl)amino)-4-oxobutyl)-1,4,7,10-tetraazacyclododecane-1,4,7-triyl)triacetic acid (p-NCS-Bz-DOTA-GA); 2,2′,2″-(10-(1-carboxy-4-((2-(2,5-dioxo-2,5-dihydro-1H-pyrrol-1-yl)ethyl)amino)-4-oxobutyl)-1,4,7,10-tetraazacyclododecane-1,4,7-triyl)triacetic acid (Maleimide-DOTA-GA); 2,2′,2″-(10-(4-((2-((((1R,8S,9s)-bicyclo[6.1.0]non-4-yn-9-ylmethoxy)carbonyl)amino)ethyl)amino)-1-carboxy-4-oxobutyl)-1,4,7,10-tetraazacyclododecane-1,4,7-triyl)triacetic acid (BCN-DOTA-GA); S-2-(4-Aminobenzyl)-diethylenetriamine pentaacetic acid (p-NH2-Bn-DTPA); S-2-(4-Isothiocyanatobenzyl)-diethylenetriamine pentaacetic acid (p-SCN-Bn-DTPA); [(R)-2-Amino-3-(4-isothiocyanatophenyl)propyl]-trans-(S,S)-cyclohexane-1,2-diamine-pentaacetic acid (p-SCN-Bn-CH-A″-DTPA); 2,2′-(1-carboxy-2-(carboxymethyl)-13-(2,5-dioxo-2,5-dihydro-1H-pyrrol-1-yl)-10-oxo-2,5,8,11-tetraazatridecane-5,8-diyl)diacetic acid (Maleimide-DTPA); 2,2′-(7-(2-((2,5-dioxopyrrolidin-1-yl)oxy)-2-oxoethyl)-1,4,7-triazonane-1,4-diyl)diacetic acid (NOTA-NHS); 2,2′-(7-(2-((2-(2,5-dioxo-2,5-dihydro-1H-pyrrol-1-yl)ethyl)amino)-2-oxoethyl)-1,4,7-triazonane-1,4-diyl)diacetic acid (Maleimide-NOTA); 2-S-(4-Isothiocyanatobenzyl)-1,4,7-triazacyclononane-1,4,7-triacetic acid (p-SCN-Bn-NOTA); 2,2′-(7-(1-carboxy-4-((2,5-dioxopyrrolidin-1-yl)oxy)-4-oxobutyl)-1,4,7-triazonane-1,4-diyl)diacetic acid (NODA-GA-NHS); 2,2′-(7-(1-carboxy-4-((2-(2,5-dioxo-2,5-dihydro-1H-pyrrol-1-yl)ethyl)amino)-4-oxobutyl)-1,4,7-triazonane-1,4-diyl)diacetic acid (Maleimide-NODA-GA); 2,2′-(7-(1-carboxy-4-((4-isothiocyanatobenzyl) amino)-4-oxobutyl)-1,4,7-triazonane-1,4-diyl)diacetic acid (p-NCS-benzyl-NODA-GA); 2,2′-(7-(4-isothiocyanatobenzyl)-1,4,7-triazonane-1,4-diyl) diacetic acid (NCS-MP-NODA); 2,2′-(7-(4-((2-((((1R,8S,9s)-bicyclo[6.1.0]non-4-yn-9-ylmethoxy)carbonyl)amino)ethyl)amino)-1-carboxy-4-oxobutyl)-1,4,7-triazonane-1,4-diyl)diacetic acid (BCN-NODA-GA); 1,4,7-Triazacyclononane-1,4-bis(acetic acid)-7-(3-azidopropylacetamide) (NO2A-Azide); 3,6,9,15-Tetraazabicyclo[9.3.1] pentadeca-1(15),11,13-triene-4-S-(4-aminobenzyl)-3,6,9-triacetic acid (p-NH2-Bn-PCTA); 3,6,9,15-Tetraazabicyclo [9.3.1]pentadeca-1(15),11,13-triene-4-S-(4-isothiocyanatobenzyl)-3,6,9-triacetic acid (p-SCN-BN-PCTA); 1-(4-isothiocyanatophenyl)-3-[6,17-dihydroxy-7,10,18,21-tetraoxo-27-(N-acetylhydroxylamino)-6,11,17,22-tetraazaheptaeicosine]thiourea (p-SCN-Bn-DFO); 2,2′-(7-(2-((2,5-dioxopyrrolidin-1-yl)oxy)-2-oxoethyl)-1,4,7-triazonane-1,4-diyl)diacetic acid (NOTA-NHS); and N-(3,11,14,22,25,33-hexaoxo-4,10,15,21,26,32-hexaaza-10,21,32-trihydroxytetratriacontane)maleimide (Maleimide-DFO). p-SCN-Bn-DFO has the following structure:

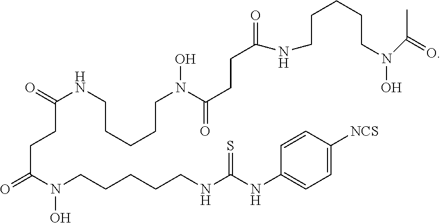

With regard to antibodies, the term “isolated” refers to an antibody that has been identified and separated and/or recovered from at least one contaminant component of its natural environment. Contaminant components may be materials that would interfere with diagnostic and/or therapeutic uses for the antibody, such as, e.g., enzymes, hormones, and/or other proteinaceous or nonproteinaceous solutes. In embodiments, the isolated antibody will be purified to greater than about 95% (such as, e.g., about 96% to about 100%) by weight of antibody as determined by the Lowry method, or to greater than about 99% by weight of antibody.

The term “monoclonal antibody” refers to an antibody obtained from a population of substantially homogeneous antibodies, wherein the individual antibodies in the population are identical except for naturally occurring post-translational modifications. Monoclonal antibodies may be highly specific, being directed against a single antigenic site. Furthermore, in contrast to polyclonal antibody preparations that include different antibodies directed against different antigenic determinants (such as, e.g., epitopes), each monoclonal antibody is directed against a single antigenic determinant on the antigen.

Monoclonal antibodies may be synthesized, and therefore may be uncontaminated by other antibodies, such as by chemical synthesis or using recombinant molecular biological synthetic techniques.

While the term monoclonal indicates the character of the antibody as being obtained from a substantially homogeneous population of antibodies, it is not intended to be construed as requiring production of the antibody by any particular method. For example, the monoclonal antibodies to be used in accordance with the present disclosure may be prepared by the Kohler hybridoma method (see e.g., Kohler et al. (1975) Nature 256:495), or they may be prepared by recombinant DNA methods (see e.g., U.S. Pat. Nos. 4,816,567 and 5,807,715, each of which is hereby incorporated by reference in its entirety). Further, monoclonal antibodies may be isolated from phage antibody libraries using standard techniques (see e.g., Clackson et al. (1991) Nature, 352:624-628 and Marks et al. (1991) J. Mol. Biol., 222:581-597).

The terms “antibody fragment” and “fragment” refer to a portion of a full length antibody that includes a region capable of recognizing and binding to a specific antigen, such as, e.g., the antigen binding, variable, or hypervariable (also known as complementarity determining) region thereof. Examples of antibody fragments include Fab, Fab′, F(ab′)₂, F(ab)₂, Fv, sFv, and scFv fragments; diabodies; linear antibodies; minibodies (see e.g., Olafsen et al. (2004) Protein Eng. Design & Sel. 17(4):315-323), a single domain antibody (sdAb, also known as a nanobody), fragments produced by a Fab expression library, anti-idiotypic (hereinafter, “anti-Id”) antibodies, complementary determining region (hereinafter, “CDR”) and epitope-binding fragments of any of the above that immunospecifically recognize and bind to IL-12, single-chain antibody molecules, and multispecific antibodies formed from antibody fragments.

The term “radiolabel” refers to a radionuclide moiety that can be attached to an antibody and that functions to provide a detectable signal, such as, e.g., radionuclide tracers. Radionuclide tracers include radioisotopes, which by virtue of radioactive decay, are detectable.

Radionuclide tracers may be detected by various imaging techniques, such as, e.g., PET and single-photon emission computed tomography (hereinafter, “SPECT”) imaging. In embodiments, radionuclide tracers have an energy of from about 20 to about 4,000 kiloelectronvolts (i.e., keV). Suitable radionuclide tracers include radionuclides that emit gamma radiation, positrons, or a combination of gamma radiation and positrons. In illustrative, non-limiting embodiments, radionuclide tracers have appropriate decay characteristics for optimizing image resolution and quantitative accuracy and/or have appropriate residualization. For example, radionuclide tracers may have a physical half-life (i.e., t_{1/2}) compatible with the time required for the antibody to achieve optimal specific:non-specific binding ratios. Examples of radionuclide tracers suitable for PET imaging, and suitable for inclusion in a labeled antibody conjugate according to embodiments of the present disclosure, include ¹¹C (t_{1/2} about 20 min), ¹³N (t_{1/2} about 10 min), ¹⁵O (t_{1/2} about 2 min), ¹⁸F (t_{1/2} about 1.83 hours), ⁴⁴Sc (t_{1/2} about 3.97 hours), ⁴⁵Ti (t_{1/2} about 3 hours), ⁵²Mn (t_{1/2} about 5.6 days), ⁶⁴Cu (t_{1/2} about 12.7 hours), ⁶⁸Ga (t_{1/2} about 1.13 hours), ⁷⁶Br (t_{1/2} about 16.2 hours), ⁸²Rb (t_{1/2} about 1.27 min), ⁸⁶Y (t_{1/2} about 14.7 hours), ⁸⁹Zr (t_{1/2} about 78.4 hours), ¹²⁴I (t_{1/2} about 100.3 hours), or a combination of any two or more thereof. Examples of radionuclide tracers suitable for SPECT imaging include ^{99m}Tc (t_{1/2} about 6 h hours), ¹¹¹In (t_{1/2} about 2.80 days), ¹³¹I (t_{1/2} about 8 days), ¹⁷⁷Lu (t_{1/2} about 6.64 days), ¹⁸⁶Re (t_{1/2} about 3.7186 days), or a combination of any two or more thereof.

The term “therapeutic radionuclide” refers to a radionuclide moiety that can be attached to an antibody that functions to deliver a cytotoxic dose of radiation, such as, e.g., a radionuclide therapeutic agent, to a target of interest, such as, e.g., a tumor. Suitable radionuclide therapeutic agents include radionuclides that emit beta particle radiation, alpha particle radiation, Auger electron radiation, or a combination of any two or more thereof. Examples of suitable radionuclide therapeutic agents include ¹²⁵I, ¹³¹I, ¹⁷⁷Lu, ¹⁸⁶Re, ²²⁵Ac, ²²⁵Ra, or a combination of any two or more thereof.

The term “immunoimaging” refers to imaging IL-12 in vivo in a subject via detection of a labeled anti-IL-12 antibody conjugate to produce an image indicative of the location and/or level of IL-12 in the subject, wherein the location and/or level of IL-12 in the subject is indicative of IL-12-producing activated APCs. Immunoimaging is used according to embodiments of the present disclosure to detect and/or monitor conditions and/or treatments involving immune activation in a subject including tumor immunology, injury, inflammatory conditions, autoimmune conditions, and infection.

Embodiments of the present disclosure are directed toward antibody conjugates, including labeled-antibody conjugates and therapeutic radionuclide-antibody conjugates, to methods for imaging, to mouse models, and to methods for assessing the effect of a composition or immune-mediated treatment for conditions involving immune activation in a subject including tumor immunology, injury, inflammatory conditions, autoimmune conditions, and infection, particularly cancer, injury, inflammatory conditions, autoimmune conditions, infection, or two or more thereof, in a human subject.

I. Antibody Conjugates

In one or more embodiments, the disclosure relates to anti-IL-12 antibody conjugates. In embodiments, anti-IL-12 antibody conjugates include both labeled-antibody conjugates and therapeutic radionuclide-antibody conjugates.

In embodiments, labeled-antibody conjugates include an antibody that specifically recognizes and binds to IL-12, and at least one detection label is conjugated to the antibody, wherein the at least one detection label includes a radionuclide tracer, a fluorophore, a nanoparticle, or any two or more thereof.

In embodiments, therapeutic radionuclide-antibody conjugates include an antibody that specifically recognizes and binds to IL-12, and at least one therapeutic radionuclide is conjugated to the antibody, wherein the at least one therapeutic radionuclide includes a radionuclide therapeutic agent.

Suitable antibodies that specifically recognize and bind to IL-12 (an anti-IL-12 antibody) may be prepared by standard techniques known to those of ordinary skill in the art or obtained commercially.

In embodiments, the anti-IL-12 antibody is an isolated antibody. In embodiments, the anti-IL-12 antibody is selected from a monoclonal antibody, an antibody fragment, or combination thereof. In embodiments, the anti-IL-12 antibody is a monoclonal antibody expressed by a hybridoma cell line or by CHO cells, NS0 cells, Sp2/0 cells, HEK cells, BHK cells, or PER.C6 cells.

In embodiments, the anti-IL-12 antibody is an antibody fragment. In further embodiments, the anti-IL-12 antibody is an Fab fragment.

Antibody fragments that recognize specific epitopes can be produced by standard techniques known to those of ordinary skill in the art. For example, F(ab′)₂ fragments can be produced by pepsin digestion of an anti-IL-12 antibody molecule. (See e.g., U.S. Pat. Nos. 4,036,945 and 4,331,647 and references contained therein; see also Nisonoff et al., Arch Biochem. Biophys. 89:230 (1960); Porter, Biochem. J. 73:119 (1959), Edelman et al., in Methods in Enzymology Vol. 1, page 422 (Academic Press 1967), and Coligan et al. Current Protocols in Immunology, Vol. 1, pages 2.8.1-2.8.10 and 2.10.-2.10.4 (John Wiley & Sons 1991)). Alternatively, Fab′ expression libraries can be constructed (see e.g., Huse et al., 1989, Science, 246:1274-1281) to allow rapid and easy identification of monoclonal Fab′ fragments with desired specificity.

In embodiments, an anti-IL-12 single chain Fv molecule (i.e., scFv) includes a V_{L} domain and a V_{H} domain. The V_{L} and V_{H} domains associate to form a target binding site. In embodiments, V_{L} and V_{H} domains are covalently linked by a peptide linker (i.e., L). In embodiments, a scFv molecule is denoted as either V_{L}-L-V_{H} if the V_{L} domain is the N-terminal part of the scFv molecule, or as V_{H}-L-V_{L} if the V_{H} domain is the N-terminal part of the scFv molecule. scFv molecules can be produced by standard techniques known to those of ordinary skill in the art. (See U.S. Pat. Nos. 4,704,692, 4,946,778, R. Raag and M. Whitlow, “Single Chain Fvs.” FASEB Vol 9:73-80 (1995) and R. E. Bird and B. W. Walker, Single Chain Antibody Variable Regions, TIBTECH, Vol 9:132-137 (1991)).

Other anti-IL-12 antibody fragments, such as, e.g., single domain antibody fragments, are also known to those of ordinary skill in the art. Single domain antibodies (i.e., V_{HH}) may be obtained, for example, from camels, alpacas or llamas by standard immunization techniques known to those of ordinary skill in the art. (See, e.g., Muyldermans et al., TIBS 26:230-235, 2001; Yau et al., J Immunol Methods 281:161-75, 2003; Maass et al., J Immunol Methods 324:13-25, 2007). In embodiments, the V_{HH} has potent antigen-binding capacity and can interact with novel epitopes that may be inaccessible to conventional V_{H}-V_{L} pairs. (See e.g., Muyldermans et al., 2001). In embodiments, alpaca serum IgG contains about 50% camel heavy chain only IgG antibodies (i.e., HCAbs) (see e.g., Maass et al., 2007). In embodiments, alpacas may be immunized with known antigens, such as, e.g., IL-12, and V_{HH}'s can be isolated that bind to and neutralize the target antigen (see e.g., Maass et al., 2007). PCR primers that amplify virtually all alpaca V_{HH} coding sequences have been identified and may be used to construct alpaca V_{HH} phage display libraries, which can be used for antibody fragment isolation by standard biopanning techniques well known to those of ordinary skill in the art. (See Maass et al., 2007).

In embodiments, an anti-IL-12 antibody fragment is produced by proteolytic hydrolysis of a full-length antibody and/or by expression in E. coli, CHO cells, or another host of DNA coding for the antibody fragment. In embodiments, antibody fragments can be obtained by pepsin or papain digestion of full-length antibodies by standard techniques known to those of ordinary skill in the art. For example, an anti-IL-12 antibody fragment can be produced by enzymatic cleavage of antibodies with pepsin to provide a fragment denoted F(ab′)2 of about 100 kDa. This fragment can be further cleaved using a thiol reducing agent, and optionally a blocking group for the sulfhydryl groups resulting from cleavage of disulfide linkages, to produce an Fab′ monovalent fragment of about 50 kDa. In alternative embodiments, an enzymatic cleavage using papain produces two monovalent Fab fragments and an Fc fragment directly.

In embodiments, anti-IL-12 antibody fragments may include peptides coding for a single complementary determining region (i.e., CDR). In embodiments, a CDR is a segment of the variable region of an anti-IL-12 antibody that is complementary in structure to the epitope to which the antibody binds and is more variable than the rest of the variable region. Accordingly, in embodiments, a CDR is referred to as a hypervariable region. In embodiments, a variable region includes three CDRs. In embodiments, CDR peptides can be produced by constructing genes encoding the CDR of an antibody of interest. Such genes may be prepared, for example, by using the polymerase chain reaction (i.e., PCR) to synthesize the variable region from RNA of antibody-producing cells. (See e.g., Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

According to embodiments, the antibody conjugate includes an anti-IL-12 diabody that specifically recognizes and binds to IL-12. A diabody included in a labeled-antibody conjugate is a noncovalent dimer of single-chain Fv (scFv) fragment that has the heavy chain variable (V_{H}) and light chain variable (V_{L}) regions connected by a linker, such as a peptide linker. The linker used is too short to allow pairing between the two domains on the same chain, such that the linked V_{H} and V_{L} domains are forced to pair with complementary domains of another chain, creating two antigen-binding sites. Diabodies are described more fully in, for example, EP 404,097; WO 93/11161; and Hollinger et al., Proc. Natl. Acad. Sci. USA, 90: 6444-6448 (1993). An exemplary linker is SGGGGS (SEQ ID NO:1).

According to embodiments, the antibody conjugate includes an anti-IL-12 nanobody that specifically recognizes and binds to IL-12. A nanobody included in a labeled-antibody conjugate is an antibody fragment which is a single monomeric variable antibody domain functional to selectively bind to a specific antigen, in this case, IL-12. Nanobodies are described more fully in, for example, Harmsen, M. et al., Appl. Microbiol. Biotechnol., 77(1):13-22, 2007.

Anti-IL-12 antibodies and antigen binding fragments can be produced according to well-known methods. Anti-IL-12 antibodies and antigen binding fragments can be obtained commercially, such as ustekinumab and briakinumab, both of which specifically bind to human IL-12, and the amino acid sequences of both of which are known, as disclosed in Bloch, Y. et al., Immunity 48 (1), 45-58 (2018); and Luo, J. et al., J. Mol. Biol. 402 (5), 797-812 (2010).

According to embodiments, an anti-IL-12 antibody included in a labeled antibody conjugate of the present disclosure is ustekinumab, briakinumab, or a functional variant, or fragment, of either thereof.

Conservative amino acid substitutions can be made in a specified amino acid sequence of an antibody or antibody fragment, or a nucleic acid encoding the antibody or antibody fragment, to produce functional variants. Conservative amino acid substitutions are art recognized substitutions of one amino acid for another amino acid having similar characteristics. For example, each amino acid can be described as having one or more of the following characteristics: electropositive, electronegative, aliphatic, aromatic, polar, hydrophobic, and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine, and valine; aromatic amino acids include phenylalanine, tyrosine, and tryptophan; polar amino acids include aspartate, glutamate, histidine, lysine, asparagine, glutamine, arginine, serine, threonine, and tyrosine; and hydrophobic amino acids include alanine, cysteine, phenylalanine, glycine, isoleucine, leucine, methionine, proline, valine, and tryptophan; and conservative substitutions include substitutions among amino acids within each group. Amino acids can also be described in terms of steric effects or relative size, e.g., alanine, cysteine, aspartate, glycine, asparagine, proline, threonine, serine, and valine are all typically considered to be small.

To determine the percent identity of two amino acid sequences, the sequences are aligned for optimal comparison purposes (e.g., gaps can be introduced in a first amino acid or nucleotide sequence for optimal alignment with a second amino acid or nucleotide sequence using the default parameters of an alignment software program). The amino acids at corresponding amino acid positions are then compared. When a position in the first sequence is occupied by the same amino acid as the corresponding position in the second sequence, then the sequences are identical at that position. The percent identity between the two sequences is a function of the number of identical positions shared by the sequences (i.e., % identity=number of identical aligned positions÷total number of aligned positions·100%). In some embodiments, the two sequences have the same length.

The determination of percent identity between two sequences can be accomplished using a mathematical algorithm. A non-limiting example of a mathematical algorithm utilized for the comparison of two sequences is the algorithm of Karlin and Altschul, 1990, PNAS USA 87:2264-68, e.g., as modified as in Karlin and Altschul, 1993, PNAS USA 90:5873-77. In calculating percent identity, only exact matches are typically counted.

Other methods of cleaving antibodies, such as, e.g., separation of heavy chains to form monovalent light-heavy chain fragments, further cleavage of fragments, or other enzymatic, chemical or genetic techniques may also be used, so long as the anti-IL-12 antibody fragments specifically bind to the antigen that is recognized by the intact antibody.

Conjugation

A detection label or radionuclide therapeutic agent is conjugated directly, or indirectly, to an anti-IL-12 antibody, or anti-IL-12 antibody fragment, to produce a labeled antibody conjugate or therapeutic radionuclide antibody conjugate according to embodiments of the present disclosure.

According to embodiments, broadly described, direct conjugation of the detection label or therapeutic radionuclide to an anti-IL-12 antibody, or anti-IL-12 antibody fragment, according to embodiments of the present disclosure includes reaction of a functional group of the detection label or therapeutic radionuclide, or a compound containing the detection label or therapeutic radionuclide, with a corresponding functional group of the anti-IL-12 antibody, or anti-IL-12 antibody fragment, such as a terminal amino group, a terminal carboxyl group or a functional group of an amino acid side chain, thereby covalently bonding the detection label or therapeutic radionuclide with the anti-IL-12 antibody, or anti-IL-12 antibody fragment.

According to embodiments, broadly described, indirect conjugation of the detection label or therapeutic radionuclide to an anti-IL-12 antibody, or anti-IL-12 antibody fragment, according to embodiments of the present disclosure includes reaction of a functional group of a linker, prosthetic group, or chelator with a corresponding functional group of the anti-IL-12 antibody, or anti-IL-12 antibody fragment, such as a terminal amino group, a terminal carboxyl group or a functional group of an amino acid side chain, thereby covalently bonding the linker, prosthetic group, or chelator with the antibody, or anti-IL-12 antibody fragment, and binding of the detection label or therapeutic radionuclide to the linker or chelator.

Non-limiting examples of functional groups which react with sulfhydryl groups of cysteine-containing antibodies or antibody fragments include epoxide, haloacetyl, and maleimide. Non-limiting examples of functional groups which react with amino groups of an antibody or antibody fragment include N-hydroxysuccinimidyl esters, carbodiimides, aldehydes, ketones, glyoxals, imidoesters, isothiocyanates, sulfonyl chlorides and acyl azides. Non-limiting examples of functional groups which react with carboxylic acid groups of an antibody or antibody fragment include amines, hydrazides, carbodiimides, diazoalkanes, diazoacetyls and carbonyldiimidazole. Additional functional groups and exemplary conjugation reactions are known in the art as exemplified in G. T. Hermanson, Bioconjugate Techniques, 2nd Edition, Academic Press, 2008.

For indirect conjugation of a detection label or therapeutic radionuclide to an antibody or antibody fragment, a suitable linker, prosthetic group, or chelator can be used, wherein the linker, prosthetic group, or chelator is bound to both the antibody, or antibody fragment, and the detection label or therapeutic radionuclide. According to particular embodiments, the linker, prosthetic group, or chelator is bifunctional and therefore functional to bind to both the antibody, or antibody fragment, and the detection label or therapeutic radionuclide, producing a labeled antibody conjugate or therapeutic radionuclide conjugate.

Linkers, prosthetic groups, chelators, and methods of use thereof for conjugation of a detection label or radionuclide therapeutic agent to an antibody, or antibody fragment, are well-known in the art, see for example, Shan S. Wong et al., Chemistry of Protein and Nucleic Acid Cross-Linking and Conjugation, Second Edition, CRC Press, 2011.

In embodiments, the labeled-antibody conjugates include at least one detection label conjugated to the antibody, or antibody fragment, wherein the at least one detection label includes a radionuclide tracer. In embodiments, the at least one detection label is a fluorophore. In embodiments, the at least one detection label is a nanoparticle. In embodiments, the at least one detection label is a metal-containing nanoparticle. Particular nanoparticles include, but are not limited to, carbon-, gold-, or iron-containing nanoparticles.

In embodiments, labeled-antibody conjugates are not limited with respect to the number of labels included. In embodiments, labeled-antibody conjugates include 1, 2, 3, 4, 5, 6, 7, 8, 9, 10 labels, or more. Where multiple labels are included, they may be the same or different.

In embodiments, the radionuclide tracer is selected from: ¹¹C, ¹³N, ¹⁵O, ¹⁸F, ⁴⁴Sc, ⁴⁵Ti, ⁵²Mn, ⁶⁴Cu, ⁶⁸Ga, ⁴⁴Sc, ⁷⁶Br, ⁸²Rb, ⁸⁶Y, ⁸⁹Zr, ⁹⁰Y, ^{99m}Tc, ¹¹¹In, ¹²⁴I, ¹³¹I, ⁴³K, ⁵²Fe, ⁵⁷Co, ⁶⁷Cu, ⁶⁷Ga, ⁷⁷Br, ⁸¹Rb, ^{81m}Kr, ^{87m}Sr, ⁸⁹Zr, ^{113m}In, ¹²³I, ¹²⁵I, ¹²⁷Cs, ¹²⁹Cs, ¹³²I, ¹⁷⁷Lu, ¹⁸⁶Re, ¹⁹⁷Hg, ²⁰³Pb, ²⁰⁶Bi, ⁸²Sr, ¹⁸⁸Re, ⁶⁰Cu, ⁶¹Cu, ⁶²Cu, ²²⁵Ac, ²²⁵Ra, and a combination of any two or more thereof.

In illustrative, non-limiting embodiments for positron emission tomography (PET), the radionuclide tracer is ⁶⁸Ga, ¹⁸F, ⁴⁴Sc, ⁶⁴Cu, ⁸⁶Y, ¹²⁴I, or ⁸⁹Zr. In illustrative, non-limiting embodiments for single positron emission computed tomography (SPECT), the radionuclide tracer is ⁶⁷Ga, ¹¹¹In, ¹⁷⁷Lu, ¹⁸⁶Re, ¹⁸⁸Re, ⁹⁰Y or ^{99m}Tc. In illustrative, non-limiting embodiments, the radionuclide tracer is ⁸⁹Zr.

In embodiments wherein the detection label is a radionuclide tracer, the radionuclide tracer is optionally conjugated to the antibody, or antibody fragment, via a bifunctional chelator.

In embodiments wherein the radionuclide tracer is ¹¹C, ¹³N, ¹⁸F, ⁷⁶Br ¹²³I, ¹²⁴I, or ¹²⁵I, a bifunctional chelator is not required for conjugation to the antibody, or antibody fragment, wherein ¹¹C, ¹³N, ¹⁸F, ⁷⁶Br ¹²³I, ¹²⁴I, or ¹²⁵I can be directly conjugated to the antibody, or antibody fragment, with standard techniques known to those of ordinary skill in the art. In non-limiting examples, a Bolton Hunter reagent (N-hydroxysuccinimide ester of mono- or di-iodinated hydroxyphenylpropionic acid), lactoperoxidase, or chloramine-T are reagents used in well-known methods of conjugating an ¹²³I, ¹²⁴I, ¹²⁵I or ¹³¹I radionuclide to an antibody.

In embodiments, the bifunctional chelator includes a chelator attached to a first reactive functional group. Suitable chelators and reactive functional groups are known to those of ordinary skill in the art. In embodiments, the bifunctional chelator includes a chelator selected from: 1,4,7-Triazacyclononane (TACN); 1,4,7,10-Tetraazacyclododecane (Cyclen); 1,4,7,10-Tetraazacyclododecane-1,7-diacetic acid (DO2A); 1,4,7,10-Tetraazacyclododecane-1,4,7-triacetic acid trisodium salt (DO3A); 1,4,7,10-Tetraazacyclododecane-1,4,7,10-tetraacetic acid (DOTA); 1,4,7,10-Tetraazacyclododecane-1,4,7,10-tetra(methylene phosphonic acid) (DOTP); 1,4,8,11-Tetraazacyclotetradecane-1,4,8,11-tetraacetic acid (TETA); 2,2′,2″,2′″-(1,4,8,11-tetraazacyclotetradecane-1,4,8,11-tetrayl)tetraacetamide (TETAM); 1,4,7,10-Tetrakis (carbamoylmethyl)-1,4,7,10-tetraazacyclododecane (DOTAM); 2,2′,2″-(1,4,7,10-tetraazacyclododecane-1,4,7-triyl)triacetamide (DO3AM); 1,8-Diamino-3,6,10,13,16,19-hexaazabicyclo [6,6,6]-eicosane (DiAmSar); 1,4,8,11-Tetraazabicyclo[6.6.2]hexadecane (CB-Cyclam); 2,2′-(1,4,8,11-tetraazabicyclo[6.6.2]hexadecane-4,11-diyl)diacetic acid (CB-TE2A); 1,4,7-Triazacyclononane-1,4,7-triacetic acid (NOTA); 1,4,7-Triazacyclononane-1,4,7-tri(methylene phosphonic acid) (NOTP); 3-(((4,7-bis ((hydroxy(hydroxymethyl)phosphoryl)methyl)-1,4,7-triazonan-1-yl) methyl)(hydroxy)phosphoryl)propanoic acid (NOPO); 2,2′,2″-(1,4,7-triazacyclononane-1,4,7-triyl)triacetamide (NOTAM); 2,2′,2″,2′″-((((carboxymethyl)azanediyl)bis(ethane-2,1-diyl))bis(azanetriyl))tetraacetic acid (DTPA); 3,6,9,15-Tetraazabicyclo[9.3.1]pentadeca-1(15),11,13-triene-3,6,9-triacetic acid (PCTA); 2,2′,2″,2′″-(1,4,7,10-tetraazacyclotridecane-1,4,7,10-tetrayl)tetraacetic acid (TRITA); 2,2′,2″,2′″-(1,4,7,10-tetraazacyclotridecane-1,4,7,10-tetrayl)tetraacetamide (TRITAM); 2,2′,2″-(1,4,7,10-tetraazacyclotridecane-1,4,7-triyl)triacetamide (TRITRAM); and 3,3′,3″-(((1,4,7-triazonane-1,4,7-triyl)tris(methylene))tris(hydroxyphosphoryl))tripropanoic acid (TRAP); or a combination of any two or more thereof. In illustrative, non-limiting embodiments, the bifunctional chelator is p-SCN-Bn-DFO.

In embodiments, the bifunctional chelator is attached to the antibody, or antibody fragment. In further embodiments, the radionuclide tracer is complexed to the bifunctional chelator, wherein the bifunctional chelator is covalently attached to the antibody, or antibody fragment.

In embodiments, the bifunctional chelator is attached to the antibody, or antibody fragment, by reacting the reactive functional group of the bifunctional chelator with a corresponding reactive group of the antibody, or antibody fragment, by standard techniques known to those of ordinary skill in the art.

In embodiments, an ion of the radionuclide tracer is complexed to the bifunctional chelator by standard techniques known to those of ordinary skill in the art.

In illustrative, non-limiting embodiments, (1) the bifunctional chelator p-SCN-Bn-DFO is reacted with the anti-IL-12 antibody, or antibody fragment, at a ratio of about 5:1 in saline solution at about pH 9 for about 1 hour at about 37° C. for covalent attachment thereof; and (2) the radionuclide tracer ⁸⁹Zr⁴⁺ is reacted with the bifunctional chelator p-SCN-Bn-DFO covalently attached to the antibody, or antibody fragment, at about pH 7.0-7.2 for about 1 hour at room temperature to form a radiolabeled-antibody conjugate.

In embodiments, a spacer can be used as linker between the detection label and the anti-IL-12 antibody, or anti-IL-12 antibody fragment. In embodiments, the spacer has a 4 to 24 carbon atom backbone.

In embodiments, a polyethylene glycol (PEGn) spacer (n=4-24) can be used as linker between the detection label and the anti-IL-12 antibody, or anti-IL-12 antibody fragment.

In embodiments, the at least one detection label includes a fluorophore. In embodiments, the fluorophore is a fluorescent dye. In embodiments, the fluorophore emits fluorescence in the visible (i.e., 400-700 nm) or near-infrared (i.e., 700-1400 nm) region.

In embodiments, the antibody conjugates include therapeutic radionuclide-antibody conjugates wherein a radionuclide therapeutic agent is bound to an anti-IL-12 directly or indirectly.

In embodiments, therapeutic radionuclide-antibody conjugates include an antibody, or antibody fragment, that specifically recognizes and binds IL-12, a bifunctional chelator conjugated to the antibody, or antibody fragment, and a radionuclide therapeutic agent complexed to the bifunctional chelator. In embodiments, the antibody, or antibody fragment, is as described herein with regard to labeled-antibody conjugates. Additionally, in embodiments, the bifunctional chelator is as described herein with regard to labeled-antibody conjugates.

In embodiments, the therapeutic radionuclide-antibody conjugate includes a radionuclide therapeutic agent complexed to the bifunctional chelator. In embodiments, the radionuclide therapeutic agent is selected from ¹²⁵I, ¹³¹I, ¹⁷⁷Lu, ¹⁸⁶Re, ²²⁵Ac, ²²⁵Ra, or a combination of any two or more thereof. In embodiments, the bifunctional chelator is attached to the antibody. In further embodiments, the radionuclide therapeutic agent is complexed to the bifunctional chelator, wherein the bifunctional chelator is covalently attached to the antibody. In embodiments, the bifunctional chelator is attached to the antibody by reacting the bifunctional chelator with the antibody. Then, in embodiments, an ion of the radionuclide therapeutic agent is complexed to the bifunctional chelator.

In embodiments, the therapeutic radionuclide-antibody conjugate includes a labeled-antibody conjugate attached to a therapeutic moiety. The therapeutic moiety can be, without limitation, a small molecule drug, an oligonucleotide or polynucleotide, such as an antisense oligonucleotide or polynucleotide, an siRNA, an mRNA, an miRNA, an shRNA, a peptide or protein. According to embodiments, a therapeutic moiety is attached to, enclosed in or partially enclosed in, a particle.

An included particle can be selected from among a lipid particle; a polymer particle; an inorganic particle; and an inorganic/organic particle. A mixture of particle types can also be included. The particles can be of any shape, size, composition, or physiochemical characteristics compatible with administration to a subject. The particles can be organic or inorganic particles, such as glass or metal and can be particles of a synthetic or naturally occurring polymer, such as polystyrene, polycarbonate, silicon, nylon, cellulose, agarose, dextran, and polyacrylamide.

In particular aspects, the particle is a lipid particle including, but not limited to, liposomes, micelles, unilamellar or multilamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486. Further description of liposomes and methods relating to their preparation and use may be found in Liposomes: A Practical Approach (The Practical Approach Series, 264), V. P. Torchilin and V. Weissig (Eds.), Oxford University Press; 2nd ed., 2003.

An included particle is typically formulated such that the particle has a diameter, or longest dimension, in the range of about 1 nm-10 microns. In particular aspects, an included particle is a nanoparticle, formulated such that the nanoparticle has a diameter, or longest dimension, in the range of about 1 nm—about 1000 nm. Further aspects of nanoparticles are described in S. M. Moghimi et al., FASEB J. 2005, 19, 311-30; Choi, et al., Mol Imaging. 2010 December; 9(6): 291-310; and Bogart et al., ACS Nano, 2014, 8 (4), pp 3107-3122.

Pharmaceutical Compositions

A pharmaceutical composition according to aspects of the present disclosure includes a labeled-antibody conjugate in an amount in the range of about 0.1-99% and a pharmaceutically acceptable carrier.

A pharmaceutical composition of the present disclosure may be in any dosage form suitable for administration to a subject, illustratively including solid, semi-solid and liquid dosage forms such as tablets, capsules, powders, granules, pills, solutions, suspensions, and gels. Liposomes and emulsions are well-known types of pharmaceutical formulations that can be used to deliver a composition of the present disclosure. Pharmaceutical compositions of the present disclosure generally include a pharmaceutically acceptable carrier such as an excipient, diluent and/or vehicle. Delayed release formulations of compositions and delayed release systems, such as semipermeable matrices of solid hydrophobic polymers can be used.

The term “pharmaceutically acceptable carrier” refers to a carrier which is suitable for use in a subject without undue toxicity or irritation to the subject and which is compatible with other ingredients included in a pharmaceutical composition.

Pharmaceutically acceptable carriers, methods for making pharmaceutical compositions and various dosage forms, as well as modes of administration are well-known in the art, for example as detailed in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005; and J. G. Hardman et al., Goodman & Gilman's The Pharmacological Basis of Therapeutics, McGraw-Hill Professional, 10th ed., 2001.

A solid dosage form for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alginates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

A composition for parenteral administration may be formulated as an injectable liquid. Liquid dosage forms for injection include one or more active agents and a pharmaceutically acceptable carrier formulated as an emulsion, solution, or suspension. A liquid dosage form for injection including a composition of the present disclosure may further include a stabilizer, a wetting agent, an emulsifying agent, a suspending agent or any two or more thereof. Examples of suitable aqueous and nonaqueous pharmaceutically acceptable carriers include water, a buffer, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

II. Methods for Imaging

In one or more embodiments, methods for imaging are disclosed herein. In embodiments, methods for imaging include: (a) administering a labeled-antibody conjugate to a subject, wherein the labeled-antibody conjugate includes: an antibody that specifically recognizes and binds to IL-12, and at least one detection label conjugated to the antibody, wherein the at least one detection label includes a radionuclide tracer, a fluorophore, a nanoparticle, or a combination or any two or more thereof; and (b) detecting the presence of the labeled-antibody conjugate in the subject in vivo by imaging. In embodiments, the labeled-antibody conjugate is as described herein with regard to antibody conjugates. In further embodiments, the antibody and the at least one detection label are as described herein with regard to antibody conjugates.

In embodiments, the methods for imaging include detecting the presence of the labeled-antibody conjugate in the subject in vivo by imaging. In embodiments, the presence of the labeled-antibody conjugate is detected in real time. In embodiments, the presence of the labeled-antibody conjugate is detected non-invasively and/or minimally-invasively.

According to aspects of the present disclosure, an antibody conjugate is an imaging agent which can be used to visualize IL-12, such as in diagnostic procedures as well as for localization of IL-12-producing activated APCs. Imaging can be performed by many procedures well-known to those having ordinary skill in the art, for example, by positron emission tomography (PET), single photon emission computed tomography (SPECT), Cerenkov imaging, photoacoustic imaging, ultrasound imaging, optical coherence tomography, optical imaging, including fluorescence imaging, magnetic resonance imaging, magnetic particle imaging, bioluminescence imaging, or a combination of any two or more thereof.

In embodiments, the methods include administering a labeled-antibody conjugate to a subject. In embodiments, the methods include administering an effective amount of a labeled-antibody conjugate to a subject. The labeled-antibody conjugate may be administered by any suitable route known to those of ordinary skill in the art. In embodiments, administration of the labeled-antibody conjugate is by intravenous, intraarterial, intraperitoneal, intramuscular, subcutaneous, intrapleural, intrathecal, intratumoral, perfusion through a regional catheter, and/or direct intralesional injection. In embodiments wherein the labeled-antibody conjugate is administered by injection, the administration may be by continuous infusion, by single bolus, and/or by multiple boluses. In some embodiments, administering the radiolabeled-antibody conjugate is non-immunogenic to the subject. According to embodiments, administering a labeled-antibody conjugate to a subject includes administering a pharmaceutical composition including the labeled-antibody conjugate and a pharmaceutically acceptable carrier.

In embodiments, the subject is a mammal. In some embodiments, the subject is a mammal selected from: humans, non-human primates, canines, felines, murines, bovines, equines, caprines, ovines, porcines, and lagomorphs. According to particular embodiments, the subject is a rodent, including, but not limited to, a rat mouse, or guinea pig. According to particular embodiments, the subject is a mouse or a human.

In embodiments, the labeled-antibody conjugate administered to a subject includes a radionuclide tracer. In some embodiments, the labeled-antibody conjugate administered to a subject includes a labeled-antibody conjugate wherein the label is a radionuclide tracer only, i.e. without a fluorophore or nanoparticle. In some embodiments, the labeled-antibody conjugate administered to a subject includes a labeled-antibody conjugate wherein the label is a fluorophore only, i.e. without a radionuclide tracer or nanoparticle. In some embodiments, the labeled-antibody conjugate administered to a subject includes a labeled-antibody conjugate wherein the label is a nanoparticle only, i.e. without a radionuclide tracer or fluorophore.

In embodiments, the radionuclide tracer is selected from: ¹¹C, ¹³N, ¹⁵O, ¹⁸F, ⁴⁴Sc, ⁴⁵Ti, ⁵²Mn, ⁶⁴Cu, ⁶⁸Ga, ⁴⁴Sc, ⁷⁶Br, ⁸²Rb, ⁸⁶Y, ⁸⁹Zr, ⁹⁰Y, ^{99m}Tc, ¹¹¹In, ¹²⁴I, ¹³¹I, ⁴³K, ⁵²Fe, ⁵⁷Co, ⁶⁷Cu, ⁶⁷Ga, ⁷⁷Br, ⁸¹Rb, ^{81m}Kr, ^{87m}Sr, ⁸⁹Zr, ^{113m}In, ¹²³I, ¹²⁵I, ¹²⁷Cs, ¹²⁹Cs, ¹³²I, ¹⁷⁷Lu, ¹⁸⁶Re, ¹⁹⁷Hg, ²⁰³Pb, ²⁰⁶Bi, ⁸²Sr, ¹⁸⁸Re, ⁶⁰Cu, ⁶¹Cu, ⁶²Cu, ²²⁵Ac, ²²⁵Ra, and a combination of any two or more thereof.

In embodiments, the imaging is positron emission tomography (PET), and the radionuclide tracer is ⁶⁸Ga, ¹⁸F, ⁴⁴Sc, ⁶⁴Cu, ⁸⁶Y, ⁸⁹Zr, ¹²⁴I, or ⁸⁹Zr. In illustrative, non-limiting embodiments, the radionuclide tracer is ⁸⁹Zr.

In embodiments, the imaging is single positron emission computed tomography (SPECT), and the radionuclide tracer is ⁶⁷Ga, ¹¹¹In, ¹⁸⁶Re, ¹⁸⁸Re, ⁹⁰Y, ¹⁷⁷Lu, or ^{99m}Tc.

In embodiments of methods for imaging, the labeled-antibody conjugate administered to a subject includes a fluorophore. In embodiments, the fluorophore is a fluorescent dye.

In embodiments wherein the labeled-antibody conjugate includes a radionuclide tracer, the presence of the labeled-antibody conjugate may be detected in vivo by PET imaging, SPECT imaging, or combination thereof. In embodiments wherein the radionuclide tracer is selected from ¹¹C, ¹³N, ¹⁵O, ¹⁸F, ⁶⁴Cu, ⁶⁸Ga, ⁴⁴Sc, ⁷⁶Br, ⁸²Rb, ⁸⁶Y, ⁸⁹Zr, ¹²⁴I, or a combination of any two or more thereof, the presence of the labeled-antibody conjugate may be detected in vivo by PET imaging. In illustrative, non-limiting embodiments for positron emission tomography (PET), the radionuclide tracer is ⁶⁸Ga, ¹⁸F, ⁶⁴Cu, ¹²⁴I, or ⁸⁹Zr. In embodiments wherein the radionuclide tracer is selected from ^{99m}Tc, ⁶⁷Ga, ¹¹¹In, ¹³¹I, ¹⁸⁶Re, ¹⁸⁸Re, ¹⁷⁷Lu, or a combination of any two or more thereof, the presence of the labeled-antibody conjugate may be detected in vivo by SPECT imaging. In illustrative, non-limiting embodiments for single positron emission computed tomography (SPECT), the radionuclide tracer is ⁶⁷Ga, ¹¹¹In, ¹⁷⁷Lu, or ^{99m}Tc.

In embodiments wherein the labeled antibody conjugate includes a fluorophore, the presence of the labeled-antibody conjugate may be detected in vivo by optical imaging. Various in vivo optical imaging techniques are known to those of ordinary skill in the art. (See e.g., Ntziachristos, Annu. Rev. Biomed. Eng. 2006, 8:1-33; Troyan, S. L. et al., Ann. Surg. Oncol. 16, 2943-2952 (2009); Luker, G. D. & Luker, K. E., J. Nucl. Med. 49, 1-4 (2008); Tromberg, B. J. et al., Med. Phys. 35, 2443-2451 (2008), and their potential applicability to imaging-guided diagnostic and surgical methods has been proposed in several preclinical studies; Kirsch, D. G. et al., Nat. Med. 13, 992-997 (2007); von Burstin, J. et al., Int. J. Cancer 123, 2138-2147 (2008); and U.S. Pat. No. 9,409,923). In embodiments, in vivo optical imaging is selected from confocal microscopy, planar imaging, fluorescence molecular tomography, complete projection tomography, fluorescence tomography direct imaging, two-photon in vivo imaging or a combination of any two or more thereof. In further embodiments, planar imaging is selected from epi-illumination (i.e., photographic) imaging, trans-illumination imaging, tomographic imaging, or a combination of any two or more thereof.

In embodiments wherein the presence of the labeled-antibody conjugate is detected by in vivo optical imaging, the fluorophore may be selected such that it emits fluorescence in the visible or near-infrared region. In illustrative, non-limiting embodiments, fluorophores emitting fluorescence in the visible or near-infrared region may be selected from fluorescein, Cy2, Cy3, Cy3B, Cy3.5, Cy5, Cy5.5, Cy7, Texas Red® (Molecular Probes, Inc., Eugene, Oreg.), AlexaFluor® (Molecular Probes, Inc., Eugene), AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750, BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665, Rhodamine Green™-X (Molecular Probes, Inc.), Rhodamine Red™-X (Molecular Probes, Inc.), Rhodamine 6G, TMR, TAMRA™ (Applied Biosystems), or a combination of any two or more thereof.

In embodiments the presence of the labeled-antibody conjugate is detected by in vivo photoacoustic imaging. In illustrative, non-limiting embodiments, photoacoustic dyes included in the conjugate may be selected from Methylene blue, Evan's blue, Trypan blue, Patent blue, Indocyanine Green, IRDye800CW, DiR, Cy7, Cy7.5, and porphyrins or a combination of any two or more thereof.

In embodiments wherein the labeled antibody conjugate includes a combination of a radionuclide tracer and a fluorophore, the presence of the labeled-antibody conjugate may be detected in vivo by a combination of PET or SPECT and optical imaging.

In embodiments wherein the labeled antibody conjugate includes a combination of a radionuclide tracer and a fluorophore, the presence of the labeled-antibody conjugate may be detected in vivo by a combination of PET or SPECT and photoacoustic imaging.

In embodiments the presence of the labeled-antibody conjugate may be detected in vivo by a combination of any two or more of: PET, SPECT, Cerenkov imaging, photoacoustic imaging, ultrasound imaging, optical coherent tomography, magnetic resonance imaging, magnetic particle imaging, optical imaging, including fluorescence imaging, and/or bioluminescence imaging.

According to embodiments, a method for imaging a subject includes 1) administering a labeled-antibody conjugate to a subject, wherein the labeled-antibody conjugate includes: an antibody that specifically binds to IL-12, and a detection label conjugated to the antibody, wherein the detection label is a radionuclide tracer, nanoparticle, and/or fluorophore; and 2) detecting the presence of the labeled-antibody conjugate in the subject in vivo by imaging.

According to particular embodiments, the subject has cancer. According to particular embodiments, the cancer is bladder cancer, brain tumors, breast cancer, cervical cancer, colorectal cancer, kidney cancer, leukemia, lung cancer, lymphoma, melanoma, myeloma, pancreatic cancer, gastric cancer, intestinal cancer, liver cancer, esophageal cancer, mesothelioma cancer, endometrial cancer, ovarian cancer, head and neck cancer, bone cancer, sarcoma, cholangiocarcinoma, gall bladder cancer, testicular cancer, thyroid cancer, or prostate cancer.

According to particular embodiments, the subject has an inflammatory condition or a disease associated with inflammation, such as, but not limited to, cardiovascular inflammation, neurological inflammation, skeletal inflammation, muscular inflammation, gastrointestinal inflammation, ocular inflammation, otic inflammation, inflammatory diseases of the joints, inflammatory diseases of the brain, inflammatory diseases of the skin, inflammatory diseases of the urogenital tract, pulmonary inflammation, chronic airway inflammation, chronic obstructive pulmonary disease (COPD), inflammation of the gall bladder, and inflammation of the bowel.

According to particular embodiments, the subject has an autoimmune condition.

According to particular embodiments, the subject has an inflammatory condition or a disease associated with inflammation due to infection and/or tissue injury.

According to particular embodiments, the subject has an inflammatory condition or a disease associated with inflammation selected from: adhesive capsulitis, allergy, Alzheimer's disease, axial spondyloarthritis, ankylosing spondylitis, appendicitis, arthritis, asthma, atopic dermatitis, Behçet's disease, bursitis, colitis cystica profunda, colitis ulcerosa, Crohn's disease, diabetes, dry eye, eczema, graft-versus-host disease, heart attack, hepatitis C, hidradenitis suppurativa, inflammatory bowel disease, irritable bowel syndrome, inflammatory pseudopolyps, lateral epicondylitis, lichen planus, lymphoma, multiple sclerosis, osteomyelitis, osteoarthritis, pancreatitis, polymyalgia rheumatic, primary biliary cirrhosis, pruritus, psoriasis, psoriatic arthritis, rheumatoid arthritis, sarcoidosis, scleroderma, systemic lupus erythematosus, synovitis, septicemia, septic shock, sinusitis, stroke, tenosynovitis, tendonitis, transplant rejection, traumatic brain injury, type I diabetes, ulcerative colitis, urticaria, uveitis, and vulvovaginitis.

According to embodiments, a method for imaging a subject includes 1) administering a labeled-antibody conjugate to a subject, wherein the labeled-antibody conjugate includes: an antibody that specifically binds to IL-12, and a detection label conjugated to the antibody, wherein the detection label is a radionuclide tracer, nanoparticle, and/or fluorophore; 2) detecting the presence of the labeled-antibody conjugate in the subject in vivo by imaging, thereby determining a level or location of IL-12 producing activated APCs in the subject. The subject may be treated for a disease or condition relating to the level or location of the detected IL-12 producing activated APCs.

In one or more embodiments, methods for treatment are disclosed herein. In embodiments, methods for treatment include: (a) administering a labeled-antibody conjugate to a subject, wherein the labeled-antibody conjugate includes: 1) an antibody that specifically recognizes and binds to IL-12, and 2) at least one radionuclide therapeutic agent. Optionally, the presence of the labeled-antibody conjugate including at least one radionuclide therapeutic agent in the subject is detected in vivo by imaging.

An administered labeled-antibody conjugate including at least one radionuclide therapeutic agent is effective to treat an undesired condition of the subject by destruction and/or inhibition of cells, such as tumor cells, microorganisms, and inappropriately located or inappropriately active immune cells, e.g. in an autoimmune condition in the subject.

Combinations of a labeled-antibody conjugate and at least one therapeutic agent are administered according to aspects of the present disclosure.

In some embodiments, a labeled-antibody conjugate and at least one additional active agent are administered to a subject to treat an inflammatory condition or a disease associated with inflammation in a subject in need thereof. In still further aspects, a labeled-antibody conjugate and at least two additional active agents are administered to a subject to treat an inflammatory condition or a disease associated with inflammation in a subject in need thereof

In some embodiments, a labeled-antibody conjugate and at least one additional active agent are administered to a subject to treat cancer in a subject in need thereof. In still further aspects, a labeled-antibody conjugate and at least two additional active agents are administered to a subject to treat cancer in a subject in need thereof.

The term “additional active agent” is used herein to denote a chemical compound, a mixture of chemical compounds, a biological macromolecule (such as a nucleic acid, an antibody, a protein or portion thereof, e.g., a peptide). The additional active agent can be, without limitation, a small molecule drug, an oligonucleotide or polynucleotide, such as an antisense oligonucleotide or polynucleotide, an siRNA, an mRNA, an miRNA, an shRNA, a peptide or protein. An additional active agent can be a therapeutic agent or a diagnostic agent according to embodiments.

Additional active agents which are therapeutic agents included in aspects of methods and compositions of the present disclosure include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, anti-inflammatory agents, anxiolytics, chemotherapeutic agents, diuretics, growth factors, hormones, non-steroidal anti-inflammatory agents, steroids and vasoactive agents.

Additional active agents which are therapeutic agents included in aspects of methods and compositions of the present disclosure include an immunomodulatory agent. According to embodiments, the immunomodulatory agent includes an anti-inflammatory agent. Anti-inflammatory agents include, but are not limited to, non-steroidal anti-inflammatory agents, glucocorticoids, and corticosteroids, such as, but not limited to, dexamethasone, prednisone, betamethasone, triamcinolone, prednisolone, and methylprednisone; cyclooxygenase inhibitors, 5-lipoxygenase inhibitors, leukotriene receptor antagonists; and metformin.

According to embodiments, combination treatments include: (1) administration of pharmaceutical compositions that include a labeled antibody conjugate in combination with one or more additional active agents; (2) co-administration of a labeled antibody conjugate with one or more additional active agents wherein the labeled antibody conjugate and the one or more additional active agents are formulated in the same composition and (3) co-administration of a labeled antibody conjugate with one or more additional active agents wherein the labeled antibody conjugate and the one or more additional therapeutic agents have not been formulated in the same composition. When using separate formulations, the labeled antibody conjugate and the one or more additional active agents may be administered at the same time or at different times.

An adjunct anti-cancer treatment can be administered in combination with a labeled antibody conjugate, such as prior to, simultaneous with or after administration of the labeled antibody conjugate, such as an anti-cancer radiation treatment of a subject or an affected area of a subject's body.

An additional active agent can be an anti-cancer agent according to embodiments, such as a targeted therapy agent or chemotherapeutic.

Anti-cancer agents are described, for example, in Goodman et al., Goodman and Gilman's The Pharmacological Basis of Therapeutics, 8th Ed., Macmillan Publishing Co., 1990.

Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

According to embodiments, the additional active agent is an anti-cancer immunotherapy agent.

According to embodiments of the present disclosure, the immunotherapy agent is an immune checkpoint inhibitor, a receptor agonist, a cytokine, a vaccine, an adoptive cell transfer therapy, an oncolytic virus, or any therapeutic wherein the mechanism of action of the therapeutic is an increased number of tumor-infiltrating lymphocytes in the subject and/or an increased activation state of a tumor-infiltrating lymphocyte population in the subject. According to embodiments, the immune checkpoint inhibitor is an inhibitor of a negative regulatory signaling pathway including PD-1, PD-L1, CTLA-4, TIM-3, or LAG-3.

According to embodiments, the immune checkpoint inhibitor selected from atezolizumab, avelumab, durvalumab, ipilimumab, nivolumab, pembrolizumab, and an antigen-binding fragment of any one of the foregoing.

According to embodiments, the receptor agonists are antibodies or other molecules inducing activation of stimulatory receptors including 4-1BB, CD27, CD40, GITR, ICOS, or OX40.

According to embodiments, cytokine therapies may include IL-2, IFN-α, or IL-15.

Vaccine platforms may include protein, peptide, recombinant vectors such as viruses or DNA, whole tumor cells with or without engineered immune stimulatory modifications, or dendritic cell vaccines.

Adoptive cell therapies include chimeric antigen receptor (CAR) T cells, expanded tumor infiltrating cells, and T cells engineered to express specific T cell receptors.

Oncolytic viral therapies include the engineered herpes simplex virus type I encoding granulocyte-macrophage colony-stimulating factor (GM-CSF) talimogene laherparepvec (T-VEC).

According to embodiments, an anti-cancer immunotherapy agent is administered to a subject and a labeled antibody conjugate is administered simultaneously or subsequently followed by detecting the presence of the labeled-antibody conjugate in the subject in vivo by imaging to image IL-12 and thereby monitor the effectiveness of the treatment with the anti-cancer immunotherapy agent. According to embodiments, the subject received an anti-cancer immunotherapy prior to administering and detecting the presence of the labeled-antibody conjugate, and wherein the mechanism of action of the immunotherapy results in an increased number of tumor-infiltrating lymphocytes in the subject and/or an increased activation state of a tumor-infiltrating lymphocyte population in the subject; or wherein administering and detecting the presence of the labeled-antibody conjugate is performed prior to administration of a therapy to determine the state of active immunity in the subject. Thus, efficacy of the anti-cancer immunotherapy is monitored non-invasively and in real-time in the subject.

In embodiments, significant binding of the labeled-antibody conjugate to the IL-12 antigen indicates the presence of IL-12-producing activated APCs and/or human cancer cells. In embodiments, significant binding of the labeled-antibody conjugate to the IL-12 antigen indicates the presence of a localized immune response in the subject. In embodiments, PET imaging is quantitative wherein a correlation is determined between uptake of the radionuclide tracer quantified by radioactivity measurements of excised tissues and uptake estimated noninvasively by PET. Thus, efficacy of the anti-cancer immunotherapy is monitored non-invasively and in real-time in the subject.

According to embodiments, an anti-inflammation immunotherapy agent is administered to a subject and a labeled antibody conjugate is administered simultaneously or subsequently followed by detecting the presence of the labeled-antibody conjugate in the subject in vivo by imaging to image IL-12 and thereby monitor the effectiveness of the treatment with the anti-inflammation immunotherapy agent. According to embodiments, the subject received an anti-inflammation immunotherapy prior to administering and detecting the presence of the labeled-antibody conjugate, and wherein the mechanism of action of the immunotherapy results in an decreased number of IL-12-producing activated APCs in the subject and/or an decreased activation state of APCs in the subject; or wherein administering and detecting the presence of the labeled-antibody conjugate is performed prior to administration of a therapy to determine the state of active immunity in the subject. Thus, efficacy of the anti-inflammation immunotherapy is monitored non-invasively and in real-time in the subject.

In embodiments, significant binding of the labeled-antibody conjugate to the IL-12 antigen indicates the presence of IL-12-producing activated APCs. In embodiments, significant binding of the labeled-antibody conjugate to the IL-12 antigen indicates the presence of a localized immune response in the subject. In embodiments, PET imaging is quantitative wherein a correlation is determined between uptake of the radionuclide tracer quantified by radioactivity measurements of excised tissues and uptake estimated noninvasively by PET. In embodiments, PET imaging is quantitative wherein uptake of the radionuclide tracer is quantified by analysis of acquired images. Thus, efficacy of the anti-inflammation immunotherapy is monitored non-invasively and in real-time in the subject.

According to embodiments, methods for imaging a subject include administering a labeled-antibody conjugate to a subject, wherein the labeled-antibody conjugate comprises: an antibody that specifically binds to IL-12, and a detection label conjugated to the antibody, wherein the detection label is a radionuclide tracer, nanoparticle, or fluorophore; and detecting the presence of the labeled-antibody conjugate in the subject in vivo by imaging.

According to embodiments, a labeled-antibody conjugate is administered to a subject prior to immunotherapy treatment, wherein the labeled-antibody conjugate includes an antibody that specifically binds to IL-12, and a detection label conjugated to the antibody, wherein the detection label is a radionuclide tracer, nanoparticle, or fluorophore; and the presence of the labeled-antibody conjugate in the subject is detected in vivo by imaging to establish a baseline reading indicative of a baseline level of IL-12 to determine the state of active immunity in the subject. An immunotherapy is then administered wherein the mechanism of action of the immunotherapy results in an increased number of IL-12—producing activated APCs in the subject and/or an increased activation state of APCs in the subject. The labeled-antibody conjugate is then administered to a subject again after the administration of the immunotherapy to establish a post-treatment reading indicative of a subsequent level of IL-12 to determine the state of active immunity in the subject. The steps of administration of the immunotherapy and administration/detection of the labeled-antibody conjugate may be performed 2, 3, 4, 5, 6, 7, 8, 9, 10, or more times, over a period of treatment time in the range of 1, 2, 3, 4, 5, 6, 7, 8, 9, 10, 11, 12, 13, 14, 15, 16, 17, 18, 19, 20, 21, 22, 23, 24, or more hours, 1, 2, 3, 4, 5, 6, 7, or more days, 1, 2, 3, 4, 5, 6, 7, 8, or more weeks, 2, 3, 4, 5, 6, 7, 8, 9, 10, 11, 12, 13, 14, 15, 16, 17, 18, 19, 20, 21, 22, 23, 24, or more months, 2, 3, 4, 5, 6, 7, 8, 9, 10, 15, 20 or more years, depending on the course of the disease and the course of treatment.

According to embodiments, a labeled-antibody conjugate is administered to a subject prior to immunotherapy treatment of cancer in a subject having or suspected of having cancer, wherein the labeled-antibody conjugate includes an antibody that specifically binds to IL-12, and a detection label conjugated to the antibody, wherein the detection label is a radionuclide tracer, nanoparticle, or fluorophore; and the presence of the labeled-antibody conjugate in the subject is detected in vivo by imaging to establish a baseline reading indicative of a baseline level of IL-12 to determine the state of active immunity in the subject. An anti-cancer immunotherapy is then administered wherein the mechanism of action of the anti-cancer immunotherapy results in an increased number of tumor-infiltrating lymphocytes in the subject and/or an increased activation state of a tumor-infiltrating lymphocyte population in the subject. The labeled-antibody conjugate is then administered to a subject again after the administration of the anti-cancer immunotherapy to establish a post-treatment reading indicative of a subsequent level of IL-12 to determine the state of active immunity in the subject. The steps of administration of the anti-cancer immunotherapy and administration/detection of the labeled-antibody conjugate may be performed 2, 3, 4, 5, 6, 7, 8, 9, 10, or more times, over a period of treatment time in the range of 1, 2, 3, 4, 5, 6, 7, 8, 9, 10, 11, 12, 13, 14, 15, 16, 17, 18, 19, 20, 21, 22, 23, 24, or more hours, 1, 2, 3, 4, 5, 6, 7, or more days, 1, 2, 3, 4, 5, 6, 7, 8, or more weeks, 2, 3, 4, 5, 6, 7, 8, 9, 10, 11, 12, 13, 14, 15, 16, 17, 18, 19, 20, 21, 22, 23, 24, or more months, 2, 3, 4, 5, 6, 7, 8, 9, 10, 15, 20, or more years, depending on the course of the disease and the course of treatment.

III. Mouse Models

In one or more embodiments, the disclosure discloses mouse models.

In embodiments, the mouse models include: a mouse having a human cancer xenograft and a labeled-antibody conjugate, wherein the labeled-antibody conjugate includes: an antibody that specifically recognizes and binds to IL-12, and at least one detection label conjugated to the antibody, wherein the at least one detection label is selected from a radionuclide tracer or a fluorophore. In embodiments, the labeled-antibody conjugate is as described herein with regard to antibody conjugates and methods of imaging.

In embodiments, the mouse model has a human cancer xenograft. In embodiments, the human cancer xenograft is transplanted into the mouse. In embodiments, the human cancer xenograft is established from human cancer cells, human cancer tissues, or combination thereof. In embodiments, the human cancer xenograft includes human cancer cells and/or human cancer tissues of primary cancer and/or metastatic cancer. In embodiments, the human cancer xenograft includes human cancer cells and/or human cancer tissues of primary cancer and/or metastatic cancer selected from brain cancer, melanoma, colon cancer, kidney cancer, prostate cancer, breast cancer, gastric cancer, pancreatic cancer, ovarian cancer and testicular cancer.

In embodiments, the mouse model having a human cancer xenograft is immunodeficient and further includes engrafted human hematopoietic stem cells such that the mouse has a “humanized” immune system which produces human IL-12.

In embodiments, the methods include determining the effect of the composition or treatment on the growth of a human cancer xenograft in the mouse. In embodiments, the effect of the composition or treatment on the growth of the human cancer xenograft is determined by imaging. In embodiments, suitable imaging techniques include those described herein with regard to imaging methods. In embodiments, the effect of the composition or treatment is determined by comparing the growth of the human cancer xenograft in the mouse administered the composition or treatment to growth of a human cancer xenograft in a mouse not administered the composition or treatment.

In embodiments, the mouse models include: a mouse having an injury or infection and a labeled-antibody conjugate, wherein the labeled-antibody conjugate includes: an antibody that specifically recognizes and binds to IL-12, and at least one detection label conjugated to the antibody, wherein the at least one detection label is selected from a radionuclide tracer or a fluorophore. In embodiments, the labeled-antibody conjugate is as described herein with regard to antibody conjugates and methods of imaging immune-mediated treatment for injury or infection.

In embodiments, the mouse models include: an immune competent mouse having a murine cancer, such as an allograft or induced murine tumor, and a labeled-antibody conjugate, wherein the labeled-antibody conjugate includes: an antibody that specifically recognizes and binds to murine IL-12, and at least one detection label conjugated to the antibody, wherein the at least one detection label is selected from a radionuclide tracer or a fluorophore. In embodiments, the labeled-antibody conjugate is as described herein with regard to antibody conjugates and methods of imaging.

### EXAMPLES

The following non-limiting examples illustrate the methods and compositions of the present disclosure.

### Example 1

Chelators, such as desferrioxamine, functionalized with an isothiocyanate functional group are conjugated to the terminal amines of an anti-IL-12 antibody at a ratio starting from 1:1 to as much as 1:50 (antibody:chelator) in buffered solutions at about pH 9 for 30 min to 2 hours at 37° C. or overnight at 4° C.

Chelators with a maleimide functional group are conjugated to the thiols found on cysteine residues of an anti-IL-12 antibody at a ratio starting from 1:5 to as much as 1:50 (mAb:chelator) at about pH 7 for 30 min to 2 hours at 37° C. or overnight at 4° C.

Purification of the resulting antibody-chelator conjugates can be made using size exclusion chromatography or centrifugal column with a molecular weight cut-off of <50,000 kDa.

Facile ⁸⁹Zr-radiolabeling of an anti-IL-12 antibody-chelator conjugate, such as anti-IL-12 antibody-desferrioxamine conjugate, to produce a labeled-antibody conjugate proceeds in a neutral pH environment (pH about 7.0-7.2) at room temperature within 1 hour. Unbound ⁸⁹Zr is removed via centrifugation with molecular weight column filters (<50,000 kDa).

### Example 2

F-18 labeling of antibodies and fragments can be done via several approaches including conjugation via [¹⁸F]-aluminum fluoride NOTA technique and N-Succinimidyl 4-[¹⁸F]fluorobenzoate. NOTA-PEG₄-Tz is prepared by attaching NOTA-Bz-NCS to tetrazine (Tz)-PEG₄-NH₂ using equimolar ratios in DMSO/TEA solvent for 1 h at room temperature (RT). Briefly, non-carrier added ¹⁸F[F⁻] will be incubated with 40 nmol AlCl₃ in 0.4 M KHCO₃ at pH˜4 for 10 min. Equimolar NOTA-PEG₄-Tz in 3:1 acetonitrile:H₂O will be added and the solution was incubated for 15 minutes at 90° C. Purification of [¹⁸F]-AlF-NOTA-Tz was made by eluting the product through a C₁₈-Solid Phase Extraction cartridge with ethanol. Labeling of antibodies proceeds via inverse electron demand Diels-Alder bioorthogonal click reaction using Tz and transcyclooctene (TCO), which affords a rapid, milder method of labeling a protein, conserving its configuration. The antibody will be attached to transcylooctene-N-hydroxysuccinimide. Incubation of the antibody-transcylcooctene with [¹⁸F]—AlF-NOTA-Tz at RT in PBS, pH ˜ 7 for 15-60 min yields the ¹⁸F-labeled antibody.

Attachment of ¹⁸F to an antibody is also made via the N-Succinimidyl 4-[¹⁸F]fluorobenzoate ([¹⁸F]SFB) prosthetic group. This is a three step, one pot reaction where [¹⁸F]SFB attaches to the terminal amines of the antibody. Nucleophilic ¹⁸F substitution of a trimethylammonium salt is made, which was followed by hydrolysis of the resulting ethyl ester and activation with N,N,N′,N′-tetramethyl-O-(Nsuccinimidyl)uronium tetrafluoroborate. Purification is performed via preparative high performance liquid chromatography using a C18 column. Dried [¹⁸F]SFB was incubated with the antibody in HEPES buffer, pH 7.8 at room temperature for 1 h. The resulting ¹⁸F-labeled antibody is purified via a PD10 desalting column or centrifugal column filters.

### Example 3

This Example demonstrates the ability of methods and compositions according to aspects of the present disclosure to identify tumor-localized immune induction after Antigen-Presenting Cell-activating Immunotherapy (APC-activating ITx). The anti-murine IL-12 antibody clone R2.9A5 and a control non-specific rat IgG isotype were conjugated to desferrioxamine (DFO) and labeled with Zr-89 (t_{1/2}˜ 3.27 days). BALB/c mice were inoculated with TUBO cells (2×10⁵) subcutaneously on the flank. Once palpable, the tumors (n=5) were treated with intratumoral injections (3×, once every other day) of adenovirus encoding the dendritic cell maturation cytokine granulocyte macrophage-colony stimulating factor (Adv/GM-CSF) (10⁸ PFU) in 10 μL PBS or vehicle control (n=5). Adv/GM-CSF models the FDA-approved GM-CSF-encoding oncolytic viral therapy T-VEC, which is shown to recruit and differentiate dendritic cells. An IL-12 specific labeled-antibody conjugate ([⁸⁹Zr]Zr-anti-IL-12) or a non-specific labeled antibody conjugate ([⁸⁹Zr]Zr-IgG) were injected into separate mice by tail vein injection on the final treatment day, when tumor volumes were 45.1±15.4 mm³ (treated) and 36.1±14.9 mm³ (untreated, p=0.404). PET imaging was conducted 72 hours after injection of the labeled antibody conjugates and tumor tissue was immediately harvested and snap-frozen for validation of IL-12 expression.

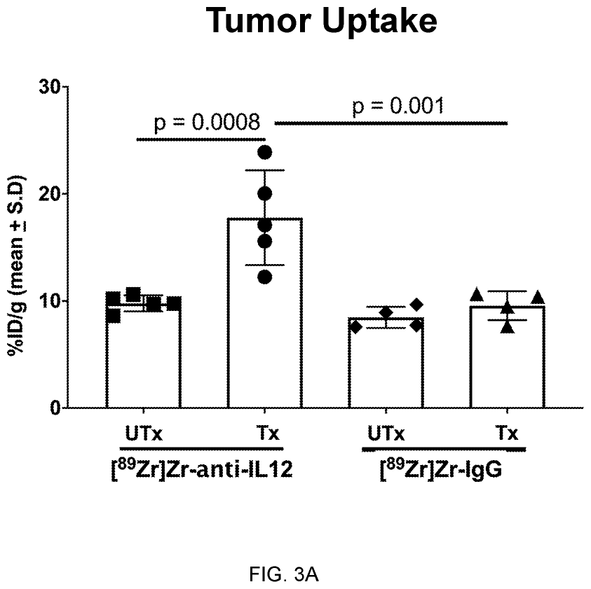

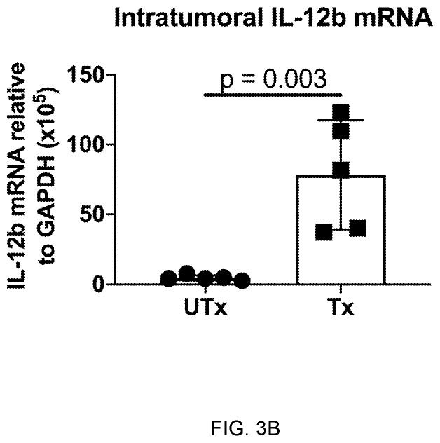

Accumulation of the IL-12 specific labeled-antibody conjugate ([⁸⁹Zr]Zr-anti-IL-12) or the non-specific labeled antibody conjugate ([⁸⁹Zr]Zr-IgG) within the tumors was reported as volumes-of-interest (VOI) expressed as % injected dose per gram of tissue (% ID/g). A higher level of accumulation of the IL-12 specific labeled-antibody conjugate was found in Adv/GM-CSF treated tumors with a VOI of 17.7±4.4% ID/g vs. untreated tumors with a VOI of 9.7±0.7% ID/g (p=0.0008) (FIG. 3A). In the groups imaged with [⁸⁹Zr]Zr-IgG, treated tumors displayed a VOI of 9.6±1.4% ID/g while untreated tumors had a VOI of 8.5±1.0% ID/g (FIG. 3A). A comparison between the uptake of [⁸⁹Zr]Zr-anti-IL-12 and [⁸⁹Zr]Zr-IgG in treated groups resulted in a statistically significant difference (p=0.001) demonstrating specificity of the IL-12 specific labeled-antibody conjugate. External validation through qRT-PCR of snap-frozen tumor tissue samples showed higher IL-12b mRNA transcription levels in treated groups (78.3±39.1) vs. control (4.7±1.8, p=0.003) (FIG. 3B).

### Example 4

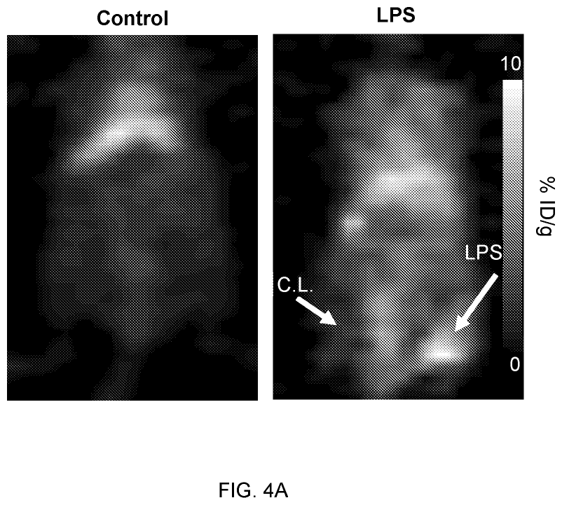

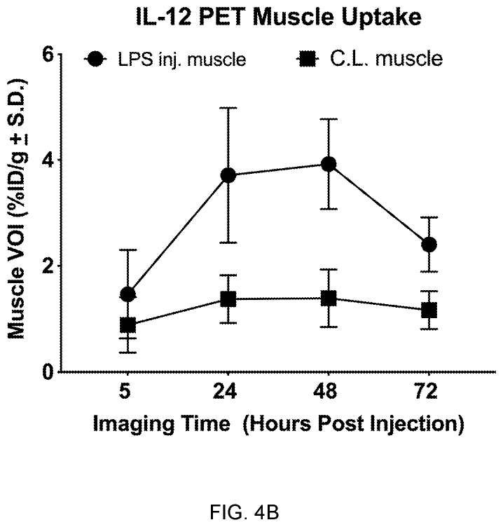

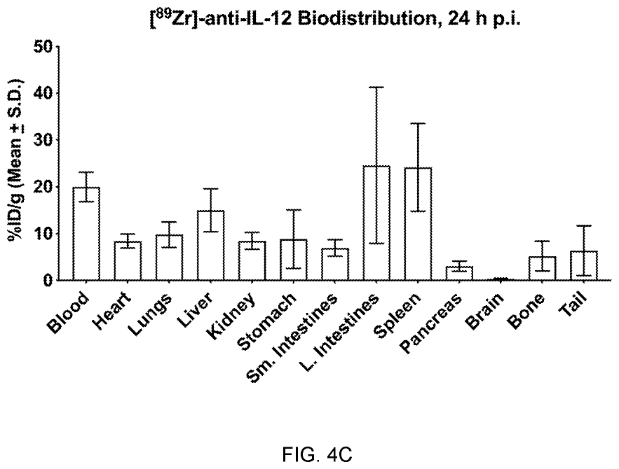

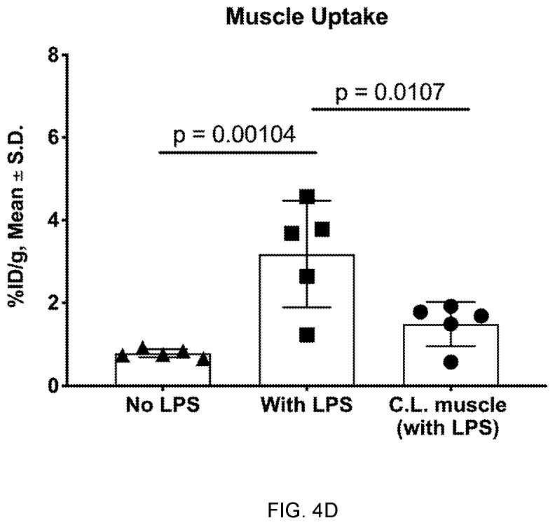

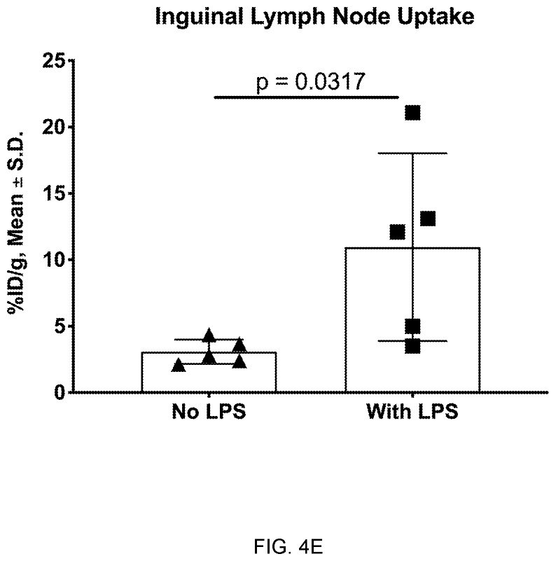

This Example demonstrates the ability of methods and compositions according to aspects of the present disclosure to identify localized IL-12 in vivo during inflammation/immune response to infection. Mice (n=5) were injected intramuscularly on the left hind leg with 40 μg lipopolysaccharide (LPS), an endotoxin of Gram-negative bacterial origin widely established to induce an acute inflammatory response. A separate naïve group of mice (n=6) was used as control. Intravenous lateral tail vein injections of [⁸⁹Zr]Zr-anti-IL-12 and of [⁸⁹Zr]Zr-IgG were administered in separate cohorts of treated and untreated mice. PET images were acquired 24-72 h post-injection (p.i.). The LPS-treated muscle demonstrated higher focal accumulation of IL-12 specific labeled-antibody conjugate ([⁸⁹Zr]Zr-anti-IL-12) compared to untreated control mice (VOI of 3.7±0.7% ID/g vs. untreated 0.7±0.1% ID/g) (FIG. 4A). The contralateral muscle of the treated groups showed lower accumulation of IL-12 specific labeled-antibody conjugate across all time points, demonstrating the specificity of the IL-12 specific labeled-antibody conjugate (FIG. 4B). Tissue distribution demonstrates pharmacokinetic properties of the IL-12 specific labeled-antibody conjugate at 24 h post injection (FIG. 4C). Comparison of the muscle uptake of the IL-12 specific labeled-antibody conjugate showed elevated IL-12 specific labeled-antibody conjugate accumulation at the injection site compared to the contralateral muscle and in a mouse with no intramuscular LPS treatment (FIG. 4D). Inguinal lymph nodes displayed higher IL-12 specific labeled-antibody conjugate uptake compared to control (FIG. 4E).

For both examples, tissue distribution of the IL-12 specific labeled-antibody conjugate in select organs was examined. Validation of the PET IL-12 specific labeled-antibody conjugate signal through qPCR and immunohistochemistry was performed.

The results demonstrate that PET imaging of the IL-12 specific labeled-antibody conjugate provided visualization of active immune response during inflammation and post-immunotherapy.

All documents cited are incorporated herein by reference; the citation of any document is not to be construed as an admission that it is prior art with respect to the present disclosure.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present disclosure have been illustrated and described, it would be obvious to one skilled in the art that various other changes and modifications can be made without departing from the spirit and scope of the disclosure. It is therefore intended to cover in the appended claims all such changes and modifications that are within the scope of this disclosure.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all technical and scientific terms used herein have the same meaning as commonly understood by one of ordinary skill in the art to which the claimed subject matter belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

1. A method for in vivo immunoimaging of IL-12 as a marker of IL-12—producing activated antigen presenting cells (APCs) in a subject, comprising:
administering a labeled-antibody conjugate to a subject, wherein the labeled-antibody conjugate comprises:
an antibody or antibody fragment that specifically binds to IL-12, and
a detection label conjugated to the antibody or antibody fragment, wherein
the labeled-antibody conjugate specifically binds to IL-12; and
detecting the presence of the labeled-antibody conjugate in the subject in vivo by imaging.

2. The method of claim 1, wherein the detection label comprises: a radionuclide tracer, a fluorophore, a nanoparticle, or any two or more thereof.

3. The method of claim 1 or 2, wherein the subject has cancer.

4. The method of claim 1, 2, or 3, wherein the subject has a condition selected from the group consisting of: an injury, an inflammatory condition, an autoimmune condition, an infection, and a combination of any two or more thereof.

5. The method of any of claims 1 to 4, wherein the subject received an immunotherapy prior to administering and detecting the presence of the labeled-antibody conjugate, and wherein the mechanism of action of the immunotherapy results in an increased number of IL-12—producing activated APCs in the subject; or wherein administering and detecting the presence of the labeled-antibody conjugate is performed prior to administration of a therapy to determine the state of active immunity in the subject.

6. The method of any one of the preceding claims, wherein the immunotherapy is an immune checkpoint inhibitor, a receptor agonist, a cytokine, a vaccine, an adoptive cell transfer therapy, an oncolytic virus.

7. The method of any one of the preceding claims, wherein the subject is human and the antibody or antibody fragment specifically binds to human IL-12.

8. The method of any one of the preceding claims, wherein the subject is a mouse and the antibody or antibody fragment specifically binds to mouse IL-12.

9. The method of any one of the preceding claims, wherein the antibody or antibody fragment is selected from: a monoclonal antibody, a monoclonal antibody fragment, or a combination thereof.

10. The method of any one of the preceding claims, wherein the antibody fragment is a diabody.

11. The method of any one of the preceding claims, wherein the radionuclide tracer is conjugated to the antibody or antibody fragment via a bifunctional chelator or linker, and wherein the bifunctional chelator, prosthetic group, or linker is attached to the antibody or antibody fragment and to the radionuclide tracer.

12. The method of claim 11, wherein the bifunctional chelator comprises a chelator selected from: 1,4,7-Triazacyclononane (TACN); 1,4,7,10-Tetraazacyclododecane (Cyclen); 1,4,7,10-Tetraazacyclododecane-1,7-diacetic acid (DO2A); 1,4,7,10-Tetraazacyclododecane-1,4,7-triacetic acid trisodium salt (DO3A); 1,4,7,10-Tetraazacyclododecane-1,4,7,10-tetraacetic acid (DOTA); 1,4,7,10-Tetraazacyclododecane-1,4,7,10-tetra(methylene phosphonic acid) (DOTP); 1,4,8,11-Tetraazacyclotetradecane-1,4,8,11-tetraacetic acid (TETA); 2,2′,2″,2′″-(1,4,8,11-tetraazacyclotetradecane-1,4,8,11-tetrayl)tetraacetamide (TETAM); 1,4,7,10-Tetrakis (carbamoylmethyl)-1,4,7,10-tetraazacyclododecane (DOTAM); 2,2′,2″-(1,4,7,10-tetraazacyclododecane-1,4,7-triyl)triacetamide (DO3AM); 1,8-Diamino-3,6,10,13,16,19-hexaazabicyclo [6,6,6]-eicosane (DiAmSar); 1,4,8,11-Tetraazabicyclo[6.6.2]hexadecane (CB-Cyclam); 2,2′-(1,4,8,11-tetraazabicyclo[6.6.2]hexadecane-4,11-diyl)diacetic acid (CB-TE2A); 1,4,7-Triazacyclononane-1,4,7-triacetic acid (NOTA); 1,4,7-Triazacyclononane-1,4,7-tri(methylene phosphonic acid) (NOTP); 3-(((4,7-bis ((hydroxy(hydroxymethyl)phosphoryl)methyl)-1,4,7-triazonan-1-yl) methyl)(hydroxy)phosphoryl)propanoic acid (NOPO); 2,2′,2″-(1,4,7-triazacyclononane-1,4,7-triyl)triacetamide (NOTAM); 2,2′,2″,2′″-((((carboxymethyl) azanediyl)bis(ethane-2,1-diyl))bis(azanetriyl))tetraacetic acid (DTPA); 3,6,9,15-Tetraazabicyclo[9.3.1]pentadeca-1(15),11,13-triene-3,6,9-triacetic acid (PCTA); 2,2′,2″,2′″-(1,4,7,10-tetraazacyclotridecane-1,4,7,10-tetrayl)tetraacetic acid (TRITA); 2,2′,2″,2′″-(1,4,7,10-tetraazacyclotridecane-1,4,7,10-tetrayl)tetraacetamide (TRITAM); 2,2′,2″-(1,4,7,10-tetraazacyclotridecane-1,4,7-triyl)triacetamide (TRITRAM); and 3,3′,3″-(((1,4,7-triazonane-1,4,7-triyl) tris(methylene))tris(hydroxyphosphoryl))tripropanoic acid (TRAP); and a combination of any two or more thereof.

13. The method of claim 11 or claim 12, wherein the bifunctional chelator is p-SCN-Bn-DFO.

14. The method of any one of the preceding claims, wherein the label comprises a radionuclide tracer selected from: ¹¹C, ¹³N, ¹⁵O, ¹⁸F, ⁴⁴Sc, ⁴⁵Ti, ⁵²Mn, ⁶⁴Cu, ⁶⁸Ga, ⁴⁴Sc, ⁷⁶Br, ⁸²Rb, ⁸⁶Y, ⁸⁹Zr, ⁹⁰Y, ^{99m}Tc, ¹¹¹In, ¹²⁴I, ¹³¹I, ⁴³K, ⁵²Fe, ⁵⁷Co, ⁶⁷Cu, ⁶⁷Ga, ⁷⁷Br, ⁸¹Rb, ^{81m}Kr, ^{87m}Sr, ⁸⁹Zr, ^{113m}In, ¹²³I, ¹²⁵I, ¹²⁷Cs, ¹²⁹Cs, ¹³²I, ¹⁷⁷Lu, ¹⁸⁶Re, ¹⁹⁷Hg, ²⁰³Pb, ²⁰⁶Bi, ⁸²Sr, ¹⁸⁸Re, ⁶⁰Cu, ⁶¹Cu, ⁶²Cu, ²²⁵Ac, ²²⁵Ra, and a combination of any two or more thereof.

15. The method of any one of the preceding claims, wherein the label comprises a radionuclide tracer selected from: ⁸⁹Zr, ¹⁸F, or both thereof.

16. The method of any one of the preceding claims, wherein the imaging comprises positron emission tomography (PET) imaging, single photon emission computed tomography (SPECT) imaging, or both PET and SPECT.

17. The method of any one of the preceding claims, wherein the labeled-antibody conjugate comprises a nanoparticle and the imaging comprises magnetic resonance imaging (MRI) and/or magnetic particle imaging (MPI).

18. The method of any one of the preceding claims, wherein the labeled-antibody conjugate comprises a fluorophore and the imaging comprises optical imaging.

19. The method of any one of the preceding claims, wherein specific binding of the labeled-antibody conjugate to IL-12 indicates the presence of IL-12—producing activated APCs.

20. The method of any one of the preceding claims, wherein the presence of the labeled-antibody conjugate is detected in real time.

21. A labeled-antibody conjugate comprising:
an antibody or antibody fragment that specifically binds to IL-12, and
a detection label conjugated to the antibody or antibody fragment, wherein the detection label is a radionuclide tracer.

22. The labeled-antibody conjugate of claim 21, wherein the detection label comprises: a radionuclide tracer, a fluorophore, a nanoparticle, or any two or more thereof.

23. The labeled-antibody conjugate of claim 22, wherein the nanoparticle is a metal-containing nanoparticle.

24. The labeled-antibody conjugate of claim 21, claim 22, or claim 23, wherein the antibody or antibody fragment is selected from a monoclonal antibody, a monoclonal antibody fragment, or combination thereof.

25. The labeled-antibody conjugate of any of claims 21 to 24, wherein the antibody fragment is an Fab′2 antibody fragment, a minibody, an ScFv antibody fragment, or a nanobody.

26. The labeled-antibody conjugate of any of claims 21 to 25 wherein the antibody fragment is a diabody.

27. The labeled-antibody conjugate of any one of claims 21 to 26, wherein the radionuclide tracer is conjugated to the antibody or antibody fragment with a bifunctional chelator, prosthetic group, or linker, and wherein the bifunctional chelator, prosthetic group, or linker is attached to the antibody or antibody fragment and to the radionuclide tracer.

28. The labeled-antibody conjugate of claim 27, wherein the bifunctional chelator comprises a chelator selected from: 1,4,7-Triazacyclononane (TACN); 1,4,7,10-Tetraazacyclododecane (Cyclen); 1,4,7,10-Tetraazacyclododecane-1,7-diacetic acid (DO2A); 1,4,7,10-Tetraazacyclododecane-1,4,7-triacetic acid trisodium salt (DO3A); 1,4,7,10-Tetraazacyclododecane-1,4,7,10-tetraacetic acid (DOTA); 1,4,7,10-Tetraazacyclododecane-1,4,7,10-tetra(methylene phosphonic acid) (DOTP); 1,4,8,11-Tetraazacyclotetradecane-1,4,8,11-tetraacetic acid (TETA); 2,2′,2″,2′″-(1,4,8,11-tetraazacyclotetradecane-1,4,8,11-tetrayl)tetraacetamide (TETAM); 1,4,7,10-Tetrakis (carbamoylmethyl)-1,4,7,10-tetraazacyclododecane (DOTAM); 2,2′,2″-(1,4,7,10-tetraazacyclododecane-1,4,7-triyl)triacetamide (DO3AM); 1,8-Diamino-3,6,10,13,16,19-hexaazabicyclo [6,6,6]-eicosane (DiAmSar); 1,4,8,11-Tetraazabicyclo[6.6.2]hexadecane (CB-Cyclam); 2,2′-(1,4,8,11-tetraazabicyclo[6.6.2]hexadecane-4,11-diyl)diacetic acid (CB-TE2A); 1,4,7-Triazacyclononane-1,4,7-triacetic acid (NOTA); 1,4,7-Triazacyclononane-1,4,7-tri(methylene phosphonic acid) (NOTP); 3-(((4,7-bis ((hydroxy(hydroxymethyl)phosphoryl)methyl)-1,4,7-triazonan-1-yl) methyl)(hydroxy)phosphoryl)propanoic acid (NOPO); 2,2′,2″-(1,4,7-triazacyclononane-1,4,7-triyl)triacetamide (NOTAM); 2,2′,2″,2′″-((((carboxymethyl) azanediyl)bis(ethane-2,1-diyl))bis(azanetriyl))tetraacetic acid (DTPA); 3,6,9,15-Tetraazabicyclo[9.3.1]pentadeca-1(15),11,13-triene-3,6,9-triacetic acid (PCTA); 2,2′,2″,2′″-(1,4,7,10-tetraazacyclotridecane-1,4,7,10-tetrayl)tetraacetic acid (TRITA); 2,2′,2″,2′″-(1,4,7,10-tetraazacyclotridecane-1,4,7,10-tetrayl)tetraacetamide (TRITAM); 2,2′,2″-(1,4,7,10-tetraazacyclotridecane-1,4,7-triyl)triacetamide (TRITRAM); and 3,3′,3″-(((1,4,7-triazonane-1,4,7-triyl) tris(methylene))tris(hydroxyphosphoryl))tripropanoic acid (TRAP); and a combination of any two or more thereof.

29. The labeled-antibody conjugate of claim 27 or claim 28, wherein the bifunctional chelator is p-SCN-Bn-DFO.

30. The labeled-antibody conjugate of any one of claims 21 to 29, wherein the radionuclide tracer is selected from: ¹¹C, ¹³N, ¹⁵O, ¹⁸F, ⁴⁴Sc, ⁴⁵Ti, ⁵²Mn, ⁶⁴Cu, ⁶⁸Ga, ⁴⁴Sc, ⁷⁶Br, ⁸²Rb, ⁸⁶Y, ⁸⁹Zr, ⁹⁰Y, ^{99m}Tc, ¹¹¹In, ¹²⁴I, ¹³¹I, ⁴³K, ⁵²Fe, ⁵⁷Co, ⁶⁷Cu, ⁶⁷Ga, ⁷⁷Br, ⁸¹Rb, ^{81m}Kr, ^{87m}Sr, ⁸⁹Zr, ^{113m}In, ¹²³I, ¹²⁵I, ¹²⁷Cs, ¹²⁹Cs, ¹³²I, ¹⁷⁷Lu, ¹⁸⁶Re, ¹⁹⁷Hg, ²⁰³Pb, ²⁰⁶Bi, ⁸²Sr, ¹⁸⁸Re, ⁶⁰Cu, ⁶¹Cu, ⁶²Cu, ²²⁵Ac, ²²⁵Ra, and a combination of any two or more thereof.

31. The labeled-antibody conjugate of any one of claims 21 to 30, wherein the radionuclide tracer is ⁸⁹Zr or ¹⁸F.

32. The labeled-antibody conjugate of any one of claims 21 to 31, wherein the antibody or antibody fragment specifically binds to human IL-12 or mouse IL-12.

33. Use of a labeled-antibody conjugate of any one of claims 21 to 32, for immunoimaging IL-12 as a marker of IL-12—producing activated antigen presenting cells (APCs).


SEQUENCE LISTING (1 sequence)

SEQ ID NO: 1 - protein, 6 aa, Artificial Sequence
  linker
SGGGGS